(19) United States
(12) Patent Application Publication
(10) Pub. No.: US 2024/0041801 A1
(43) Pub. Date: Feb. 8, 2024

(54) COMBINATION THERAPY FOR TREATING COVID-19

(71) Applicant: DrugArray, Inc., La Jolla, CA (US)

(72) Inventor: Dayu TENG, La Jolla, CA (US)

(21) Appl. No.: 18/467,230

(22) Filed: Sep. 14, 2023

Related U.S. Application Data
(63) Continuation-in-part of application No. PCT/US2022/020227, filed on Mar. 14, 2022.
Application No. PCT/US2022/020227 is a continuation-in-part of application No. PCT/US2022/020228, filed on Mar. 14, 2022.
Application No. PCT/US2022/020228 is a continuation-in-part of application No. PCT/US2022/020229, filed on Mar. 14, 2022.
(60) Provisional application No. 63/161,369, filed on Mar. 15, 2021.
(60) Provisional application No. 63/161,372, filed on Mar. 15, 2021.
(60) Provisional application No. 63/161,375, filed on Mar. 15, 2021.

(51) Int. Cl. A61K 31/155; A61K 31/675; A61K 31/708; A61K 31/7072; A61K 31/505; A61K 31/635; A61K 31/47; A61K 31/522; A61K 31/665; C07K 16/10; A61K 31/551; A61P 31/14; A61K 39/42

(52) U.S. Cl. CPC A61K 31/155; A61K 31/675; A61K 31/708; A61K 31/7072; A61K 31/505; A61K 31/635; A61K 31/47; A61K 31/522; A61K 31/665; C07K 16/1003; A61K 31/551; A61P 31/14; A61K 39/42; C07K 2317/21

(57) ABSTRACT

The present disclosure provides methods of treating coronavirus-associated diseases in an individual comprising administering to the individual an effective amount of peramivir, cidofovir, or nevirapine and an effective amount of a second antiviral agent. Also provided are methods of treating coronavirus-associated diseases in an individual comprising administering to the individual an effective amount of nevirapine and an effective amount of valacyclovir and/or nevirapine. Also provided are formulations and methods of delivering the formulations to an individual.

### CROSS-REFERENCE TO RELATED APPLICATIONS

This application is a continuation-in-part of International Patent Application Serial No. PCT/US2022/020227, entitled “COMBINATION THERAPY FOR TREATING COVID-19,” having an international filing date of Mar. 14, 2022, which claims priority to U.S. provisional patent application No. 63/161,369, filed Mar. 15, 2021. The present application is also a continuation-in-part of International Patent Application Serial No. PCT/US2022/020228, entitled “COMBINATION THERAPY FOR TREATING COVID-19,” having an international filing date of Mar. 14, 2022, which claims priority to U.S. provisional patent application No. 63/161,372, filed Mar. 15, 2021. The present application is also a continuation-in-part of International Patent Application Serial No. PCT/US2022/020229, entitled “COMBINATION THERAPY FOR TREATING COVID-19,” having an international filing date of Mar. 14, 2022, which claims priority to U.S. provisional patent application No. 63/161,375, filed Mar. 15, 2021. The disclosures and contents of the above-referenced applications are incorporated herein by reference in their entireties for all purposes.

### TECHNICAL FIELD

The present disclosure is directed to methods of treating coronavirus-associated diseases in an individual comprising administering to the individual an effective amount of peramivir, cidofovir, or nevirapine and a second antiviral agent. Also provided are compositions of peramivir, cidofovir, or nevirapine and a second antiviral agent, and methods and dosing of delivering the combination compositions to an individual.

### BACKGROUND

Coronaviruses are known to cause deadly diseases. For example, Coronavirus Disease-2019 (COVID-19), which is caused by SARS-CoV-2 (also known as 2019-nCov; a member of the coronavirus family), may lead to rapid onset of Acute Respiratory Distress Syndrome (ARDS) in addition to causing cardio-pulmonary distress. COVID-19 has a high fatality rate (about 3+%). The unique and devastating characteristics of COVID-19 are attributable to the high transmissibility of SARS-CoV-2, which is akin to the common cold. SARS-CoV-2, and the spread of COVID-19, is nearly impossible to contain due to the high transmissibility and the lengthy, and often asymptomatic, incubation period (on average about 7-14 days, and up to 20+ days in certain cases). Additionally, much like influenza, coronaviruses are RNA viruses and are prone to mutation. It has already been reported that several unique strains of SARS-CoV-2 exist. Vaccine-based approaches for mitigating the spread of COVID-19 may have very limited success due the continual mutation of the virus. Such a strategy will be in a “continuous” catch-up cycle. Other means for preventing and treating coronavirus-associated diseases are desperately needed.

### BRIEF SUMMARY

In some aspects, provided herein is a method of treating an infectious disease in an individual comprising administering to an individual a) an effective amount of peramivir and b) an effective amount of at least one other antiviral agent, wherein the other antiviral agent is selected from the group consisting of a reverse transcriptase inhibitor, a protease inhibitor, an integrase inhibitor, and a viral DNA polymerase inhibitor.

In some embodiments, the infectious disease is a coronavirus-associated disease. In some embodiments, the coronavirus-associated disease is Severe Acute Respiratory Syndrome Coronavirus 2 (COVID-19), an Angiotensin-Converting Enzyme 2 (ACE2)-associated disease, Acute Respiratory Distress Syndrome (ARDS), Severe Acute Respiratory Syndrome (SARS), or Middle East Respiratory Syndrome (MERS).

In some embodiments, the other antiviral agent is a reverse transcriptase inhibitor. In some embodiments, the reverse transcriptase inhibitor is a nucleoside analog reverse transcriptase inhibitor. In some embodiments, the nucleoside analog reverse transcriptase inhibitor is abacavir. In other embodiments, the nucleoside analog reverse transcriptase inhibitor is didanosine. In other embodiments, the nucleoside analog reverse transcriptase inhibitor is stavudine.

In some embodiments, the reverse transcriptase inhibitor is a non-nucleoside reverse transcriptase inhibitor. In some embodiments, the non-nucleoside reverse transcriptase inhibitor is etravirine. In other embodiments, the non-nucleoside reverse transcriptase inhibitor is rilpivirine.

In some embodiments, the other antiviral agent is a protease inhibitor. In some embodiments, the protease inhibitor is darunavir.

In some embodiments, the other antiviral agent is an integrase inhibitor. In other embodiments, the integrase inhibitor is elvitegravir.

In some embodiments, the other antiviral agent is a viral DNA polymerase inhibitor. In some embodiments, the viral DNA polymerase inhibitor is acyclovir. In other embodiments, the viral DNA polymerase inhibitor is ganciclovir. In other embodiments, the viral DNA polymerase inhibitor is valganciclovir.

In some embodiments, the peramivir is administered intramuscularly. In other embodiments, the peramivir is administered intravenously. In other embodiments, the peramivir is administered orally. In some embodiments, the other antiviral agent is administered orally. In other embodiments, the other antiviral agent is administered intramuscularly. In other embodiments, the other antiviral agent is administered intravenously.

In some embodiments, the peramivir is administered as a one-time dose. In some embodiments, the abacavir is administered orally twice daily. In some embodiments, the abacavir is administered orally once daily. In some embodiments, the didanosine is administered orally twice daily. In some embodiments, the stavudine is administered orally every 12 hours. In some embodiments, the etravirine is administered orally once daily. In some embodiments, the etravirine is administered orally twice daily. In some embodiments, the rilpivirine is administered rally once daily. In some embodiments, the rilpivirine is administered intramuscularly monthly. In some embodiments, the darunavir is administered orally twice daily. In some embodiments, the elvitegravir is administered orally once daily. In some embodiments, the acyclovir is administered intravenously every 8 hours for 5-10 days. In some embodiments, the ganciclovir is administered intravenously every 12 hours for 14-21 days. In some embodiments, the ganciclovir is administered orally three times per day. In some embodiments, the ganciclovir is administered orally six times per day. In some embodiments, the valganciclovir is administered orally twice daily for 21 days. In some embodiments, the acyclovir is administered orally five times a day for 5-10 days.

In some embodiments, the peramivir and the other antiviral agent are administered simultaneously. In some embodiments, the peramivir and the other antiviral agent are administered in a single composition.

In some embodiments, the peramivir and the other antiviral agent are administered sequentially. In some embodiments, the peramivir is administered prior to administration of the other antiviral agent. In other embodiments, the peramivir is administered following administration of the other antiviral agent.

In some embodiments, the individual is a human. In some embodiments, the individual suffers from a pre-existing health condition correlated with poor prognosis following SARS-CoV-2 disease. In some embodiments, the pre-existing health condition is selected from the group consisting of cancer, chronic kidney disease, chronic obstructive pulmonary disease, Down Syndrome, heart conditions, heart failure, coronary artery disease, cardiomyopathy, immunocompromised states, obesity, pregnancy, sickle cell disease, smoking, Type I diabetes mellitus, Type 2 diabetes mellitus, asthma, cerebrovascular disease, cystic fibrosis, hypertension, neurologic conditions, liver disease, pulmonary fibrosis, thalassemia, and 65 years or greater of age.

In some embodiments, a second therapy is administered to the individual. In some embodiments, the second therapy comprises remdesivir, monoclonal antibodies, mechanical ventilation, or combinations thereof. In some embodiments, the second therapy comprises administration of an effective amount of remdesivir. In some embodiments, the second therapy comprises administration of an effective amount of monoclonal antibody targeting against SARS-CoV-2. In some embodiments, the second therapy comprises administration of casirivimab and imdevimab intravenously. In some embodiments, the second therapy is mechanical ventilation.

In some aspects, provided herein is a pharmaceutical composition comprising a) peramivir and b) at least one other antiviral agent, wherein the other antiviral agent is selected from the group consisting of a reverse transcriptase inhibitor, a protease inhibitor, an integrase inhibitor, and a viral DNA polymerase inhibitor.

In some embodiments, the other antiviral agent is a reverse transcriptase inhibitor.

In some embodiments, the reverse transcriptase inhibitor is a nucleoside analog reverse transcriptase inhibitor. In some embodiments, the nucleoside analog reverse transcriptase inhibitor is abacavir. In other embodiments, the nucleoside analog reverse transcriptase inhibitor is didanosine. In other embodiments, the nucleoside analog reverse transcriptase inhibitor is stavudine.

In some embodiments, the reverse transcriptase inhibitor is a non-nucleoside reverse transcriptase inhibitor. In some embodiments, the non-nucleoside reverse transcriptase inhibitor is etravirine. In other embodiments, the non-nucleoside reverse transcriptase inhibitor is rilpivirine.

In some embodiments, the other antiviral agent is a protease inhibitor. In some embodiments, the protease inhibitor is darunavir.

In some embodiments, the other antiviral agent is an integrase inhibitor. In some embodiments, the integrase inhibitor is elvitegravir.

In some embodiments, the other antiviral agent is a viral DNA polymerase inhibitor. In some embodiments, the viral DNA polymerase inhibitor is acyclovir. In other embodiments, the viral DNA polymerase inhibitor is ganciclovir. In other embodiments, the viral DNA polymerase inhibitor is valganciclovir.

In some embodiments, the pharmaceutical composition is a tablet. In some embodiments, the pharmaceutical composition is a capsule. In some embodiments, the pharmaceutical composition is a caplet. In some embodiments, the pharmaceutical composition is in a vial.

In some embodiments, the weight ratio of the peramivir and the other antiviral in the composition is about 25:1 to about 1:5.

In some embodiments, the pharmaceutical composition contains about 100-1000 mg peramivir. In some embodiments, the pharmaceutical composition contains about 50-1000 mg abacavir. In some embodiments, the pharmaceutical composition contains about 50-1000 mg didanosine. In some embodiments, the pharmaceutical composition contains about 1-1000 mg stavudine. In some embodiments, the pharmaceutical composition contains about 25-1000 mg etravirine. In some embodiments, the pharmaceutical composition contains about 5-500 mg rilpivirine. In some embodiments, the pharmaceutical composition contains about 100-1000 mg/mL rilpivirine. In some embodiments, the pharmaceutical composition contains about 100-1000 mg darunavir. In some embodiments, the pharmaceutical composition contains about 25-500 mg elvitegravir. In some embodiments, the pharmaceutical composition contains about 25-2000 mg acyclovir. In some embodiments, the pharmaceutical composition contains about 50-2500 mg ganciclovir. In some embodiments, the pharmaceutical composition contains about 250-2000 mg valganciclovir.

In some aspects, there is provided a pharmaceutical composition according to any one of the pharmaceutical compositions described herein (e.g., including any of the combinations disclosed in FIG. 3A, FIG. 3B, and FIG. 4), for use in the manufacture of a medicament for treating or preventing an infectious disease in a subject in thereof.

In some aspects, there is provided a pharmaceutical composition according to any one of the pharmaceutical compositions provided herein (e.g., including any of the combinations disclosed in FIG. 3A, FIG. 3B, and FIG. 4), for use in treating or preventing an infectious disease in a subject in thereof.

In some aspects, there is provided use of any one of the pharmaceutical described herein (e.g., including any of the combinations disclosed in FIG. 3A, FIG. 3B, and FIG. 4) for use in treating or preventing an infectious disease in a subject in thereof.

In some aspects, provided herein is a kit comprising agents described herein (e.g., including agents in any of the combinations disclosed in FIG. 3A, FIG. 3B, and FIG. 4). In some embodiments, the agents in the kit are in separate compositions. In some aspects, provided herein is a kit, comprising: a) peramivir; b) at least one other antiviral agent, wherein the other antiviral agent is selected from the group consisting of a reverse transcriptase inhibitor, a protease inhibitor, an integrase inhibitor, and a viral DNA polymerase inhibitor; and optionally c) instructions for using a) and b) in combination for treating or preventing an infectious disease in a subject in thereof. In some embodiments, a) and b) in the kit are in separate compositions.

In some aspects, provided herein is a method of treating an infectious disease in an individual comprising administering to an individual a) an effective amount of cidofovir and b) an effective amount of at least one other antiviral agent, wherein the other antiviral agent is selected from the group consisting of a reverse transcriptase inhibitor and a viral DNA polymerase inhibitor.

In some embodiments, the infectious disease is a coronavirus-associated disease. In some embodiments, the coronavirus-associated disease is Severe Acute Respiratory Syndrome Coronavirus 2 (COVID-19), an Angiotensin-Converting Enzyme 2 (ACE2)-associated disease, Acute Respiratory Distress Syndrome (ARDS), Severe Acute Respiratory Syndrome (SARS), or Middle East Respiratory Syndrome (MERS).

In some embodiments, the other antiviral agent is a reverse transcriptase inhibitor. In some embodiments, the reverse transcriptase inhibitor is a nucleoside analog reverse transcriptase inhibitor. In some embodiments, the nucleoside analog reverse transcriptase inhibitor is zidovudine. In other embodiments, the nucleoside analog reverse transcriptase inhibitor is stavudine.

In some embodiments, the other antiviral agent is a viral DNA polymerase inhibitor. In some embodiments, the viral DNA polymerase inhibitor is valacyclovir.

In some embodiments, the cidofovir is administered orally. In other embodiments, the cidofovir is administered intramuscularly. In other embodiments, the cidofovir is administered intravenously. In some embodiments, the other antiviral agent is administered orally. In other embodiments, the other antiviral agent is administered intramuscularly. In other embodiments, the other antiviral agent is administered intravenously.

In some embodiments, the cidofovir is administered as a one-time dose. In some embodiments, the zidovudine is administered orally once daily. In some embodiments, the stavudine is administered orally every 12 hours. In some embodiments, the valacyclovir is administered orally twice daily for 10 days.

In some embodiments, the cidofovir and the other antiviral agent are administered simultaneously. In some embodiments, the cidofovir and the other antiviral agent are administered as a single composition.

In some embodiments, the cidofovir and the other antiviral agent are administered sequentially. In some embodiments, the cidofovir is administered prior to administration of the other antiviral agent. In other embodiments, the cidofovir is administered following administration of the other antiviral agent.

In some embodiments, the individual is a human. In some embodiments, the individual suffers from a pre-existing health condition correlated with poor prognosis following SARS-CoV-2 disease. In some embodiments, the pre-existing health condition is selected from the group consisting of cancer, chronic kidney disease, chronic obstructive pulmonary disease, Down Syndrome, heart conditions, heart failure, coronary artery disease, cardiomyopathy, immunocompromised states, obesity, pregnancy, sickle cell disease, smoking, Type I diabetes mellitus, Type 2 diabetes mellitus, asthma, cerebrovascular disease, cystic fibrosis, hypertension, neurologic conditions, liver disease, pulmonary fibrosis, thalassemia, and 65 years or greater of age.

In some embodiments, a second therapy is administered to the individual. In some embodiments, the second therapy comprises remdesivir, monoclonal antibodies, mechanical ventilation, or combinations thereof. In some embodiments, the second therapy comprises administration of an effective amount of remdesivir. In some embodiments, the second therapy comprises administration of an effective amount of monoclonal antibody targeting against SARS-CoV-2. In some embodiments, the second therapy comprises administration of casirivimab and imdevimab intravenously. In some embodiments, the second therapy is mechanical ventilation.

In some aspects, herein provided is a pharmaceutical composition comprising a) cidofovir and b) at least one other antiviral agent, wherein the other antiviral agent is selected from the group consisting of a reverse transcriptase inhibitor and a viral DNA polymerase inhibitor.

In some embodiments, the other antiviral agent is a reverse transcriptase inhibitor. In some embodiments, the reverse transcriptase inhibitor is a nucleoside analog reverse transcriptase inhibitor. In some embodiments, the nucleoside analog reverse transcriptase inhibitor is zidovudine. In other embodiments, the nucleoside analog reverse transcriptase inhibitor is stavudine.

In some embodiments, the other antiviral agent is a viral DNA polymerase inhibitor. In some embodiments, the viral DNA polymerase inhibitor is valacyclovir.

In some embodiments, the pharmaceutical composition is a tablet. In some embodiments, the pharmaceutical composition is a capsule. In some embodiments, the pharmaceutical composition is a caplet. In some embodiments, the pharmaceutical composition is in a vial.

In some embodiments, the weight ratio of the cidofovir and the other antiviral in the composition is about 25:1 to about 1:5.

In some embodiments, the pharmaceutical composition contains about 25-1000 mg cidofovir. In some embodiments, the pharmaceutical composition contains about 100-1000 mg zidovudine. In some embodiments, the pharmaceutical composition contains about 1-1000 mg stavudine. In some embodiments, the pharmaceutical composition contains about 0.25 to 5 grams valacyclovir.

In some aspects, there is provided a pharmaceutical composition according to any one of the pharmaceutical compositions described herein (e.g., including any of the combinations disclosed in FIG. 3A, FIG. 3B, and FIG. 4), for use in the manufacture of a medicament for treating or preventing an infectious disease in a subject in thereof.

In some aspects, there is provided a pharmaceutical composition according to any one of the pharmaceutical compositions provided herein (e.g., including any of the combinations disclosed in FIG. 3A, FIG. 3B, and FIG. 4), for use in treating or preventing an infectious disease in a subject in thereof.

In some aspects, there is provided use of any one of the pharmaceutical described herein (e.g., including any of the combinations disclosed in FIG. 3A, FIG. 3B, and FIG. 4) for use in treating or preventing an infectious disease in a subject in thereof.

In some aspects, provided herein is a kit comprising agents described herein (e.g., including agents in any of the combinations disclosed in FIG. 3A, FIG. 3B, and FIG. 4). In some embodiments, the agents in the kit are in separate compositions. In some aspects, provided herein is a kit, comprising: a) cidofovir; b) at least one other antiviral agent, wherein the other antiviral agent is selected from the group consisting of a reverse transcriptase inhibitor, a protease inhibitor, an integrase inhibitor, and a viral DNA polymerase inhibitor; and optionally c) instructions for using a) and b) in combination for treating or preventing an infectious disease in a subject in thereof. In some embodiments, a) and b) in the kit are in separate compositions.

In some aspects, provided herein is a method of treating an infectious disease in an individual comprising administering to an individual a) an effective amount of nevirapine and b) and effective amount of a second antiviral agent, wherein the second antiviral agent is selected from the group consisting of a reverse transcriptase inhibitor, a protease inhibitor, an integrase inhibitor, and a viral DNA polymerase inhibitor.

In some aspects, provided herein is a method of treating an infectious disease in an individual comprising administering to an individual a) an effective amount of nevirapine and b) and effective amount of valacyclovir.

In some aspects, provided herein is a method of treating an infectious disease in an individual comprising administering to an individual a) an effective amount of nevirapine and b) and effective amount of etravirine.

In some embodiments, the infectious disease is a coronavirus-associated disease. In some embodiments, the coronavirus-associated disease is Severe Acute Respiratory Syndrome Coronavirus 2 (COVID-19), an Angiotensin-Converting Enzyme 2 (ACE2)-associated disease, Acute Respiratory Distress Syndrome (ARDS), Severe Acute Respiratory Syndrome (SARS), or Middle East Respiratory Syndrome (MERS).

In some embodiments, the nevirapine is administered orally. In other embodiments, the nevirapine is administered intramuscularly. In other embodiments, the nevirapine is administered intravenously. In some embodiments, the valacyclovir is administered orally. In other embodiments, the valacyclovir is administered intramuscularly. In other embodiments, the valacyclovir is administered intravenously. In some embodiments, the etravirine is administered orally. In other embodiments, the etravirine is administered intramuscularly. In other embodiments, the etravirine is administered intravenously.

In some embodiments, the nevirapine is administered orally once daily for 14 days. In some embodiments, the valacyclovir is administered orally twice daily for 10 days. In some embodiments, the etravirine is administered orally twice daily for 14 days.

In some embodiments, the nevirapine and the etravirine are administered simultaneously. In some embodiments, the nevirapine and the etravirine are administered in a single composition.

In some embodiments, the nevirapine and the valacyclovir are administered simultaneously. In some embodiments, the nevirapine and the valacyclovir are administered in a single composition.

In some embodiments, the nevirapine and the etravirine are administered sequentially. In some embodiments, the nevirapine is administered prior to administration of the etravirine. In some embodiments, the nevirapine is administered following administration of the etravirine.

In some embodiments, the nevirapine and the valacyclovir are administered sequentially. In some embodiments, the nevirapine is administered prior to administration of the valacyclovir. In some embodiments, the nevirapine is administered following administration of the valacyclovir.

In some embodiments, the individual is a human. In some embodiments, the individual suffers from a pre-existing health condition correlated with poor prognosis following SARS-CoV-2 disease. In some embodiments, the pre-existing health condition is selected from the group consisting of cancer, chronic kidney disease, chronic obstructive pulmonary disease, Down Syndrome, heart conditions, heart failure, coronary artery disease, cardiomyopathy, immunocompromised states, obesity, pregnancy, sickle cell disease, smoking, Type I diabetes mellitus, Type 2 diabetes mellitus, asthma, cerebrovascular disease, cystic fibrosis, hypertension, neurologic conditions, liver disease, pulmonary fibrosis, thalassemia, and 65 years or greater of age.

In some embodiments, a second therapy is administered to the individual. In some embodiments, the second therapy comprises remdesivir, monoclonal antibodies, mechanical ventilation, or combinations thereof. In some embodiments, the second therapy comprises administration of an effective amount of remdesivir. In some embodiments, the second therapy comprises administration of an effective amount of a monoclonal antibody targeting against SARS-CoV-2. In some embodiments, the second therapy comprises administration of casirivimab and imdevimab intravenously. In some embodiments, the second therapy is mechanical ventilation.

In some aspects, provided herein is a pharmaceutical composition comprising a) nevirapine and b) valacyclovir.

some aspects, provided herein is a pharmaceutical composition comprising a) nevirapine and b) etravirine.

In some embodiments, the pharmaceutical composition is a tablet. In some embodiments, the pharmaceutical composition is a capsule. In some embodiments, the pharmaceutical composition is a caplet. In some embodiments, the pharmaceutical composition is in a vial.

In some embodiments, the weight ratio of the nevirapine and the valacyclovir in the composition is about 25:1 to about 1:25.

In some embodiments, the weight ratio of the nevirapine and the etravirine in the composition is about 25:1 to about 1:25.

In some embodiments, the pharmaceutical composition contains about 50-1000 mg nevirapine. In some embodiments, the pharmaceutical composition contains about 0.25 to 5 grams valacyclovir.

In some embodiments, the pharmaceutical composition contains about 50-1000 mg nevirapine. In some embodiments, the pharmaceutical composition contains about 0.1 to 5 grams etravirine.

In some aspects, there is provided a pharmaceutical composition according to any one of the pharmaceutical compositions described herein (e.g., including any of the combinations disclosed in FIG. 3A, FIG. 3B, and FIG. 4), for use in the manufacture of a medicament for treating or preventing an infectious disease in a subject in thereof.

In some aspects, there is provided a pharmaceutical composition according to any one of the pharmaceutical compositions provided herein (e.g., including any of the combinations disclosed in FIG. 3A, FIG. 3B, and FIG. 4), for use in treating or preventing an infectious disease in a subject in thereof.

In some aspects, there is provided use of any one of the pharmaceutical described herein (e.g., including any of the combinations disclosed in FIG. 3A, FIG. 3B, and FIG. 4) for use in treating or preventing an infectious disease in a subject in thereof.

In some aspects, provided herein is a kit comprising agents described herein (e.g., including agents in any of the combinations disclosed in FIG. 3A, FIG. 3B, and FIG. 4). In some embodiments, the agents in the kit are in separate compositions. In some aspects, provided herein is a kit, comprising: a) nevirapine; b) at least one other antiviral agent, wherein the other antiviral agent is selected from the group consisting of a reverse transcriptase inhibitor, a protease inhibitor, an integrase inhibitor, and a viral DNA polymerase inhibitor; and optionally c) instructions for using a) and b) in combination for treating or preventing an infectious disease in a subject in thereof. In some embodiments, a) and b) in the kit are in separate compositions.

In some embodiments, the infectious disease is a coronavirus-associated disease. In some embodiments, the coronavirus-associated disease is Severe Acute Respiratory Syndrome Coronavirus 2 (COVID-19), an Angiotensin-Converting Enzyme 2 (ACE2)-associated disease, Acute Respiratory Distress Syndrome (ARDS), Severe Acute Respiratory Syndrome (SARS), or Middle East Respiratory Syndrome (MERS).

### DESCRIPTION OF THE DRAWINGS

The drawings illustrate certain features and advantages of this disclosure. These embodiments are not intended to limit the scope of the appended claims in any manner.

FIG. 1A and FIG. 1B show the effect of various antiviral agents on the growth and proliferation of SARS-CoV-2 in Vero cell culture. FIG. 1A shows antiviral agents and combinations of antiviral agents that result in a low SARS-CoV-2 density per cell from Plate 1. FIG. 1B shows antiviral agents and combinations of antiviral agents that result in a low SARS-CoV-2 density per cell from Plate 2.

FIG. 2A and FIG. 2B show the effect of various antiviral agents on the growth and proliferation of SARS-CoV-2 in Vero cell culture. FIG. 2A shows antiviral agents and combinations of antiviral agents that result in a high SARS-CoV-2 density per cell from Plate 1. FIG. 2B shows antiviral agents and combinations of antiviral agents that have a high SARS-CoV-2 density per cell from Plate 2.

FIG. 3A and FIG. 3B show the quantified average intensity of SARS-CoV-2 per cell for each antiviral combination tested. FIG. 3A shows the results for Plate 1. FIG. 3B shows the results for Plate 2.

FIG. 4 shows the quantified intensity per cell for each antiviral combination for Plates 1 and 2.

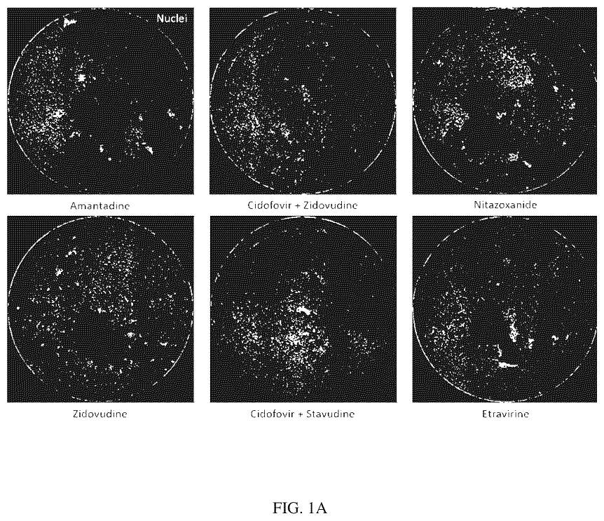

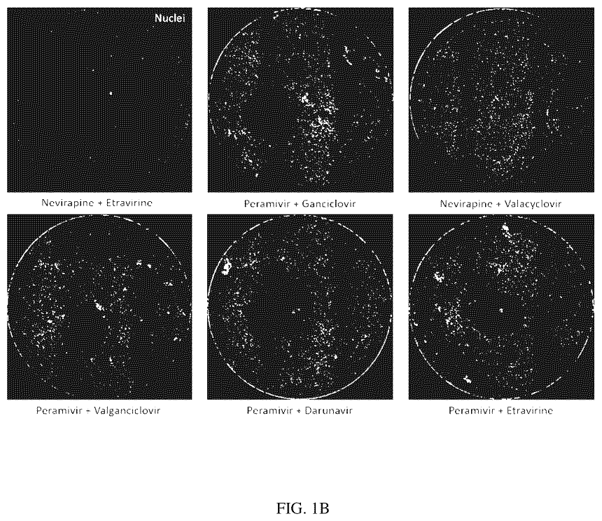

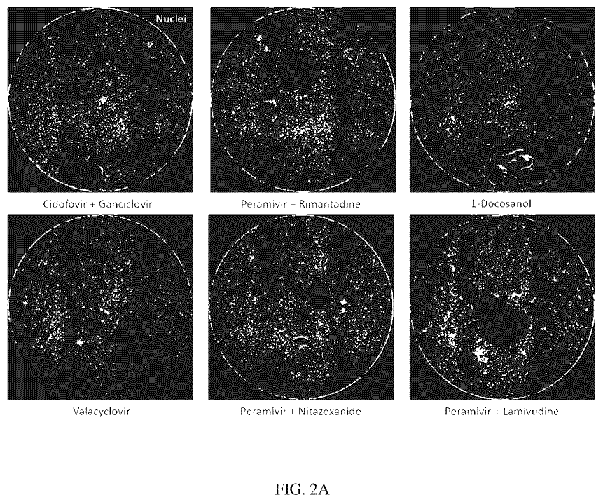

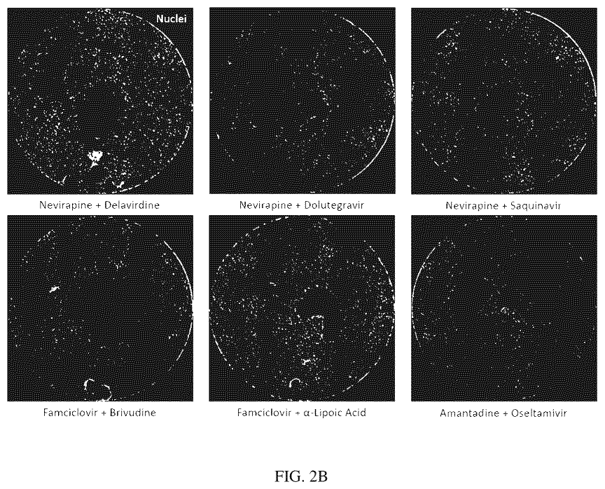

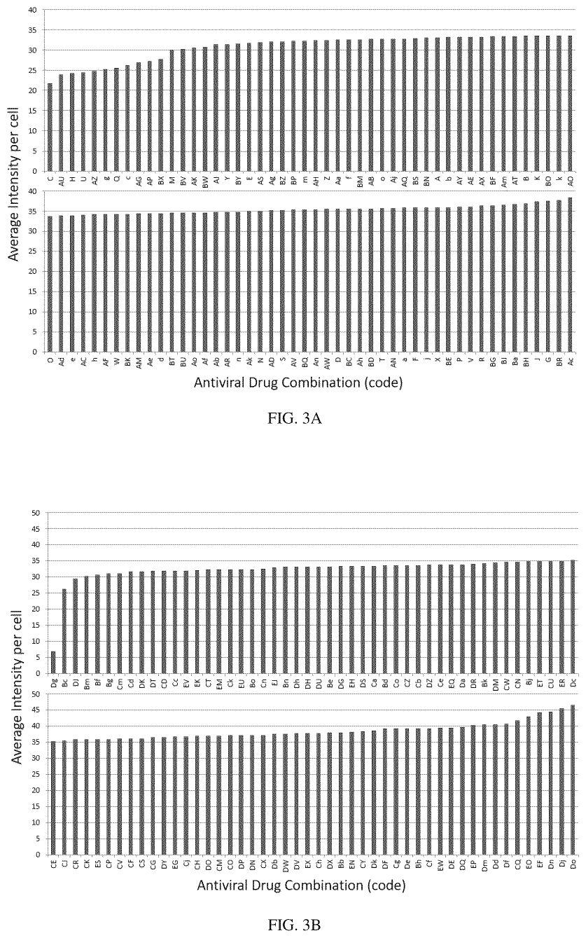

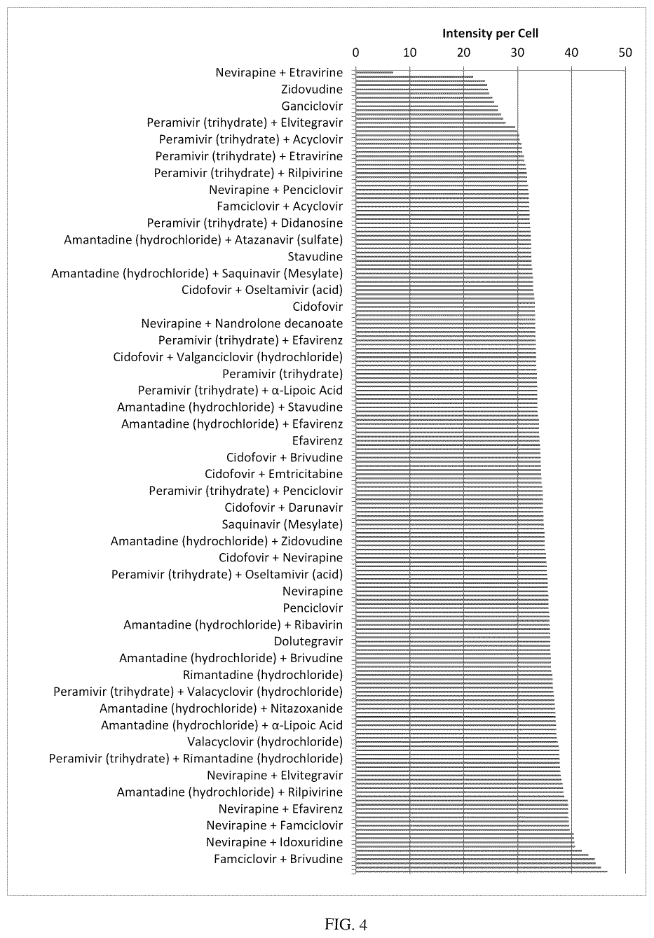

### DETAILED DESCRIPTION

All publications, comprising patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section headings used herein are for organizational purposes only and are not to be construed as limiting the subject matter described.

Provided herein, in some aspects, is a method of treating a coronavirus-associated disease in an individual, the method comprising administering to the individual an effective amount of peramivir and a second antiviral agent. The present disclosure is based on the surprising finding that certain combinations of peramivir with a second antiviral agent can be useful for treating a coronavirus-associated disease by reducing the replication and proliferation of SARS-CoV-2. The present application thus in one aspect provides a method of treating a coronavirus-associated disease in an individual comprising administering to an individual an effective amount of peramivir, and an effective amount of at least one other antiviral agent, wherein the other antiviral agent is selected from the group consisting of a reverse transcriptase inhibitor, a protease inhibitor, an integrase inhibitor, and a viral DNA polymerase inhibitor. In another aspect, there is provided a pharmaceutical composition comprising peramivir and at least one other antiviral agent, wherein the other antiviral agent is selected from the group consisting of a reverse transcriptase inhibitor, a protease inhibitor, an integrase inhibitor, and a viral DNA polymerase inhibitor.

Provided herein, in some aspects, is a method of treating a coronavirus-associated disease in an individual, the method comprising administering to the individual an effective amount of cidofovir and a second antiviral agent. The present disclosure is based on the surprising finding that certain combinations of cidofovir with a second antiviral agent can be useful for treating a coronavirus-associated disease by reducing the replication and proliferation of SARS-CoV-2. The present application thus in one aspect provides a method of treating a coronavirus-associated disease in an individual comprising administering to an individual an effective amount of cidofovir, and an effective amount of at least one other antiviral agent, wherein the other antiviral agent is selected from the group consisting of a reverse transcriptase inhibitor and a viral DNA polymerase inhibitor. In another aspect, there is provided a pharmaceutical composition comprising cidofovir and at least one other antiviral agent, wherein the other antiviral agent is selected from the group consisting of a reverse transcriptase inhibitor and a viral DNA polymerase inhibitor.

Provided herein, in some aspects, is a method of treating a coronavirus-associated disease in an individual, the method comprising administering to the individual an effective amount of nevirapine and an effective amount of a second antiviral agent. The present disclosure is based on the surprising finding that combinations of nevirapine with valacyclovir or etravirine can be useful for treating a coronavirus-associated disease by reducing the replication and proliferation of SARS-CoV-2. The present application thus in one aspect provides a method of treating a coronavirus-associated disease in an individual comprising administering to an individual an effective amount of nevirapine, and an effective amount of valacyclovir or etravirine. In another aspect, there is provided a pharmaceutical composition comprising nevirapine and valacyclovir. In yet another aspect, there is provided a pharmaceutical composition comprising nevirapine and etravirine.

### Definitions

For purpose of interpreting this specification, the following definitions will apply and, whenever appropriate, terms used in the singular will also include the plural and vice versa. In the event that any definition set forth below conflicts with any document incorporated herein by reference, the definition set forth shall control.

“Antiviral” or “antiviral agent” refers to a category of antimicrobial drugs that are used specifically for treating viral infections by inhibiting the development of the viral pathogen inside the host cell. “Antivirals” and “antiviral agents” described herein include, but are not limited to, several categories based on their target, including: 1) entry blockers, which interfere with the attachment and penetration of the virus into the host cell; 2) nucleoside/nucleotide analogues and nonnucleoside analogues, which interfere with nucleic acid synthesis by blocking viral polymerases; this class includes viral DNA polymerase inhibitors and reverse transcriptase inhibitors; 3) protein synthesis inhibitors, which interfere with viral replication; 4) protease inhibitors, which interfere with the maturation of the virus and its infectivity; and 5) integrase inhibitors. (DeClercq, Antiviral drugs in current clinical use, J. Clin. Virol., 30, 115-33, 2004). “Viral DNA polymerase inhibitor” is an antiviral agent that inhibits the function of a viral DNA polymerase required for viral replication.

“Reverse transcriptase inhibitor” is an antiviral agent that inhibits the function of a reverse transcriptase enzyme required for viral replication.

“Protein synthesis inhibitor” is an antiviral agent that inhibits the function of a protein synthesis machinery required for viral replication.

“Protease inhibitor” is an antiviral agent that inhibits the function of a protease enzyme required for viral replication.

“Integrase inhibitor” is an antiviral agent that inhibits the function of an integrase enzyme required for viral replication.

“Treating” a disease or disorder with the compounds and methods discussed herein is defined as administering one or more of the compounds discussed herein, with or without additional therapeutic agents, in order to reduce or eliminate either the disease or disorder or one or more symptoms of the disease or disorder, or to retard the progression of the disease or disorder or of one or more symptoms of the disease or disorder, or to reduce the severity of the disease or disorder or of one or more symptoms of the disease or disorder.

The term “effective amount,” as used herein, refers to an amount of a compound or composition sufficient to treat a specified disorder, condition, or disease such as ameliorate, palliate, lessen, and/or delay one or more of its symptoms. As is understood in the art, an “effective amount” may be in one or more doses, e.g., a single dose or multiple doses may be required to achieve the desired treatment endpoint. An effective amount may be considered in the context of administering one or more therapeutic agents, and combinations may be considered to be given in an effective amount if a desirable or beneficial result may be or is achieved. The components (e.g., the first and second therapies) in a combination treatment described in the present application may be administered sequentially, simultaneously, or concurrently using the same or different routes of administration for each component. Thus, an effective amount of a combination therapy includes an amount of the first therapy and an amount of the second therapy that when administered sequentially, simultaneously, or concurrently produces a desired outcome.

“In conjunction with” or “in combination with” refers to administration of one treatment modality in addition to another treatment modality. As such, “in conjunction with” or “in combination with” refers to administration of one treatment modality before, during or after delivery of the other treatment modality to the individual.

The term “simultaneous administration,” as used herein, means that a first agent and second agent in a combination therapy are administered with a time separation of no more than about 15 minutes, such as no more than about any of 10, 5, or 1 minutes. When the first and second agents are administered simultaneously, the first and second gents may be contained in the same composition (e.g., a composition comprising both a first and second agent) or in separate compositions (e.g., a first agent is contained in one composition and a second agent is contained in another composition).

As used herein, the term “sequential administration” means that the first agent and second agent in a combination therapy are administered with a time separation of more than about 15 minutes, such as more than about any of 20, 30, 40, 50, 60, or more minutes. Either the first agent or the second agent may be administered first. The first and second agents are contained in separate compositions, which may be contained in the same or different packages or kits.

A “subject,” “individual,” or “patient” is a vertebrate. In some embodiments, the vertebrate is a mammal. In other embodiments, the subject, individual, or patient is a food animal, such as a chicken, turkey, duck, goose, cow, lamb, sheep, pig, or goat. In other embodiments, the subject, individual, or patient is a domestic animal, such as a cat, dog, bird, rabbit, or guinea pig. The compounds, compositions, and methods disclosed herein can be used in human medicine and in veterinary medicine. In some embodiments, the individual is a human.

The term “pharmaceutically acceptable,” as used herein, is meant a material that is not biologically or otherwise undesirable, e.g., the material may be incorporated into a pharmaceutical composition administered to a patient without causing any significant undesirable biological effects or interacting in a deleterious manner with any of the other components of the composition in which it is contained. Pharmaceutically acceptable carriers, excipients, or salts have preferably met the required standards of toxicological and manufacturing testing and/or are included on the Inactive Ingredient Guide prepared by the U.S. Food and Drug administration.

The terms “comprising,” “having,” “containing,” and “including,” and other similar forms, and grammatical equivalents thereof, as used herein, are intended to be equivalent in meaning and to be open ended in that an item or items following any one of these words is not meant to be an exhaustive listing of such item or items, or meant to be limited to only the listed item or items. For example, an article “comprising” components A, B, and C can consist of (i.e., contain only) components A, B, and C, or can contain not only components A, B, and C but also one or more other components. As such, it is intended and understood that “comprises” and similar forms thereof, and grammatical equivalents thereof, include disclosure of embodiments of “consisting essentially of” or “consisting of.”

Where a range of values is provided, it is understood that each intervening value, to the tenth of the unit of the lower limit, unless the context clearly dictates otherwise, between the upper and lower limit of that range and any other stated or intervening value in that stated range, is encompassed within the disclosure, subject to any specifically excluded limit in the stated range. Where the stated range includes one or both of the limits, ranges excluding either or both of those included limits are also included in the disclosure.

Reference to “about” a value or parameter herein includes (and describes) variations that are directed to that value or parameter per se. For example, description referring to “about X” includes description of “X.” In some embodiments, numerical designations are provided herein for ease of understanding the scope of the present disclosure, wherein the numerical designations are calculated from experimental values and may include approximations, e.g., rounded weight percentages calculated from an amount of a starting material. In some embodiments, numerical designations provided herein, e.g., weight percentages, may vary (±) by increments of 0.1 to 0.5.

Reference to “about” a value or parameter herein includes (and describes) variations that are directed to that value or parameter per se. For example, description referring to “about X” includes description of “X.”

The term “about X-Y” used herein has the same meaning as “about X to about Y.” As used herein, including in the appended claims, the singular forms “a,” “or,” and “the” include plural referents unless the context clearly dictates otherwise.

Treatment with Peramivir

Methods

Provided herein are methods for treating and/or preventing coronavirus-associated infection in an individual comprising administering to the individual an effective amount of peramivir and a second antiviral agent. In some embodiments, the second antiviral agent is a reverse transcriptase inhibitor. In other embodiments the other antiviral agent is a protease inhibitor. In other embodiments, the other antiviral agent is an integrase inhibitor. In other embodiments, the other antiviral agent is a viral DNA polymerase inhibitor.

Certain aspects of the present disclosure relate to viral infections. In some embodiments, the virus is an enveloped virus. Examples of enveloped viruses are well known in the art and include, without limitation, the virus families of Arenavirus, Arterivirus, Asfarvirus, Baculovirus, Bunyavirus, Coronavirus, Cystovirus, Deltavirus, Filovirus, Flavivirus, Fusellovirus, Hepadnavirus, Herpesvirus, Iridovirus, Lipothrixivirus, Orthomyxovirus, Paramyxovirus, Plasmavirus, Polydnavirus, Poxvirus, Retrovirus, Rhabdovirus, and Togavirus. In some embodiments, the virus is a Coronavirus, e.g., severe acute respiratory syndrome coronavirus 1 (SARS-CoV-1), severe acute respiratory syndrome coronavirus 2 (SARS-CoV-2), or Middle East respiratory syndrome-related coronavirus (MERS-CoV). In some embodiments, the virus is an Orthomyxovirus, e.g., influenza virus A, B, or C. In some embodiments, the virus is an Orthopneumovirus, e.g., respiratory syncytial virus (RSV).

In some embodiments, an individual has been diagnosed with a coronavirus-associated disease. In some embodiments, an individual has been diagnosed with Severe Acute Respiratory Syndrome Coronavirus 2 (COVID-19). In some embodiments, an individual has or has been diagnosed with acute respiratory distress syndrome (ARDS), e.g., prior to treatment with the combination therapy. Methods for diagnosis of ARDS include, without limitation, chest X-ray, CT scanning, and/or measurement of oxygen levels. In some embodiments, an individual has been diagnosed with an Angiotensin-Converting Enzyme 2 (ACE2)-associated disease. In some embodiments, an individual has been diagnosed with Severe Acute Respiratory Syndrome (SARS). In some embodiments, an individual has been diagnosed with Middle East Respiratory Syndrome (MERS).

In some embodiments, an individual is treated with a combination of peramivir and another antiviral agent. In some embodiments, the other antiviral agent is a reverse transcriptase inhibitor. In some embodiments, the reverse transcriptase inhibitor is a nucleoside analog reverse transcriptase inhibitor. In some embodiments the nucleoside analog reverse transcriptase inhibitor is abacavir. In some embodiments the nucleoside analog reverse transcriptase inhibitor is didanosine. In some embodiments, the nucleoside analog reverse transcriptase inhibitor is stavudine. In other embodiments, the reverse transcriptase inhibitor is a non-nucleoside reverse transcriptase inhibitor. In some embodiments, the non-nucleoside reverse transcriptase inhibitor is etravirine. In other embodiments, the non-nucleoside reverse transcriptase inhibitor is rilpivirine.

In some embodiments the other antiviral agent is a protease inhibitor. In some embodiments, the protease inhibitor is darunavir. In some embodiments, the other antiviral agent is an integrase inhibitor. In some embodiments, the integrase inhibitor is elvitegravir. In some embodiments, the other antiviral agent is a DNA polymerase inhibitor. In some embodiments, the DNA polymerase inhibitor is acyclovir. In some embodiments the DNA polymerase inhibitor is ganciclovir. In other embodiments, the DNA polymerase inhibitor is valganciclovir.

In some embodiments, the other antiviral agent is administered orally. In some embodiments, the other antiviral agent is administered intramuscularly. In other embodiments, the other antiviral agent is administered intravenously. In some embodiments, the peramivir and the other antiviral agent are administered simultaneously.

In some embodiments, the peramivir and other antiviral agent are administered in a single composition. In some embodiments, the single composition is administered orally. In some embodiments, the single composition is administered intravenously. In some embodiments, the single composition is administered intramuscularly.

The composition comprising peramivir and the other antiviral agent can be administered simultaneously (i.e., simultaneous administration and/or sequentially (i.e., sequential administration)).

In some embodiments, the peramivir and the other antiviral agent are administered simultaneously. The term “simultaneous administration,” as used herein, means that the peramivir and the other antiviral agent are administered with a time separation no more than about 15 minutes(s), such as no more than about any of 10, 5, or 1 minutes. When the drugs are administered simultaneously, the peramivir and the other antiviral compound may be contained in the same composition (e.g., a composition comprising the peramivir and the other antiviral agent) or in separate compositions (e.g., the peramivir is contained in one composition and the other antiviral agent is contained in another composition).

In some embodiments, the peramivir and the other antiviral agent are administered sequentially. The term “sequential administration” as used herein means that the peramivir and the other antiviral agent are administered with a time separation of more than about 15 minutes, such as more than any of 20, 30, 40, 50, or more minutes. Either the peramivir or the other antiviral agent may be administered first. In some embodiments, the peramivir is administered prior to administration of the other antiviral agent. In some embodiments, the peramivir is administered following administration of the other antiviral agent. The peramivir and the other antiviral agent are contained in separate compositions, which may be contained in the same or different packages.

In some embodiments, the individual is a human. In some embodiments, the individual suffers from a pre-existing health condition correlated with poor prognosis following SARS-CoV2-disease. In some embodiments, the pre-existing health condition is cancer. In some embodiments, the pre-existing health condition is chronic kidney disease. In some embodiments, the pre-existing health condition is chronic obstructive pulmonary disease. In some embodiments, the pre-existing health condition is Down Syndrome. In some embodiments, the pre-existing health condition is a heart condition. In some embodiments, the pre-existing health condition is heart failure. In some embodiments, the pre-existing health condition is coronary artery disease. In some embodiments, the pre-existing health condition is cardiomyopathy. In some embodiments, the pre-existing health condition is an immunocompromised state. In some embodiments, the pre-existing health condition is obesity. In some embodiments, the pre-existing health condition is pregnancy. In some embodiments, the pre-existing health condition is sickle cell disease. In some embodiments, the pre-existing health condition is smoking. In some embodiments, the pre-existing health condition is Type I diabetes mellitus. In some embodiments, the pre-existing health condition is Type 2 diabetes mellitus. In some embodiments, the pre-existing health condition is asthma. In some embodiments, the pre-existing health condition is cerebrovascular disease. In some embodiments, the pre-existing health condition is cystic fibrosis. In some embodiments, the pre-existing health condition is hypertension. In some embodiments, the pre-existing health condition is a neurologic condition. In some embodiments, the pre-existing health condition is liver disease. In some embodiments, the pre-existing health condition is pulmonary fibrosis. In some embodiments, the pre-existing health condition is thalassemia. In some embodiments, the pre-existing health condition is 65 years or greater of age.

In some embodiments, the peramivir and other antiviral is accompanied by a second therapy administered to the individual. In some embodiments, the second therapy is remdesivir. In some embodiments, the second therapy is monoclonal antibody. In some embodiments, the monoclonal antibody is targeted against SARS-CoV-2. In some embodiments, the second therapy is casirivimab and imdevimab. In some embodiments, the second therapy is administered intravenously. In some embodiments, the second therapy is mechanical ventilation. Peramivir

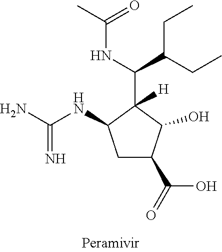 [SMILES: CCC(CC)[C@H](NC(C)=O)[C@@H]1[C@H](O)[C@@H](C(=O)O)C[C@H]1NC(=N)N]

Peramivir is an antiviral compound that belongs to the class of neuraminidase inhibitors. Neuraminidase inhibitors interfere with the normal processing of viral particles preventing viral particles from being released from infected cells. Peramivir is an inhibitor of the influenza neuraminidase enzyme and is used as therapy of acute symptomatic influenza A and B. Peramivir has not been associated with serum enzyme elevations during therapy or with clinically apparent liver injury.

Peramivir is injectable and has been approved for the treatment of acute and uncomplicated influenza in patients 2 years of age and older who have been symptomatic for no more than two days. Peramivir is approved to treat Type A and B influenza. In some embodiments, the peramivir is administered intravenously. In other embodiments, the peramivir is administered intramuscularly. In other embodiments, the peramivir is administered orally. In some embodiments, the individual is administered a one-time dose. In some embodiments, the individual is administered more than one dose. In some embodiments, the individual is administered 600 mg peramivir. In some embodiments, the individual is administered 100, 200, 300, 400, 500, 600, 700, 800, 900, or 1000 mg peramivir. In some embodiments, the individual is administered 1500 or 2000 mg peramivir or more.

Reverse Transcriptase Inhibitors

Reverse transcriptase inhibitors are a class of anti-viral drug. In particular, the class is used to treat HIV/AIDS. Reverse transcriptase inhibitors inhibit the activity of reverse transcriptase, an enzyme required for the replication of retroviruses.

Suitable reverse transcriptase inhibitors described herein include, for example, 1) nucleoside analog reverse transcriptase inhibitors (NARTIs or NRTIs); 2) non-nucleoside reverse-transcriptase inhibitors (NNRTIs); 3) nucleoside reverse transcriptase translocation inhibitors (NNRTIs); and 4) Portmanteau inhibitors.

In some embodiments, the other antiviral agent is a reverse transcriptase inhibitor. In some embodiments, the reverse transcriptase inhibitor is a nucleoside analog reverse transcriptase inhibitor. Nucleoside analog reverse transcriptase inhibitors are first converted into an active compound through intracellular phosphorylation to a triphosphate form. The triphosphate form of the nucleoside analog inhibitor competes with cellular nucleotides and inhibits the reverse transcriptase enzyme by introducing a chain terminator into the growing DNA strand during reverse transcription. In some embodiments, the other antiviral is a nucleoside analog reverse transcriptase inhibitor. In some embodiments, the nucleoside analog reverse transcriptase inhibitor is abacavir. In some embodiments, the nucleoside analog reverse transcriptase inhibitor is didanosine. In some embodiments, the nucleoside reverse transcriptase inhibitor is stavudine.

In some embodiments, the nucleoside analog reverse transcriptase inhibitor is a thymidine analogue. In some embodiments, the thymidine analogue is stavudine. In some embodiments, the nucleoside analogue reverse transcriptase inhibitor is a cytidine analogue. In some embodiments, the nucleoside analogue reverse transcriptase inhibitor is a guanosine analogue. In some embodiments, the guanosine analogue is abacavir. In some embodiments, the nucleoside analog reverse transcriptase inhibitor is an adenosine analogue. In some embodiments, the adenosine analogue is didanosine.

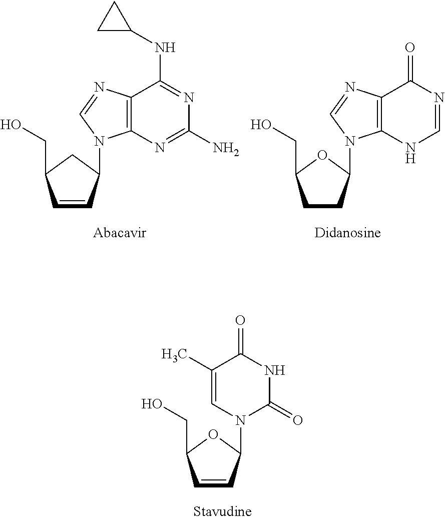

In some embodiments, the reverse transcriptase inhibitor is a non-nucleoside reverse transcriptase inhibitor. Non-nucleoside reverse transcriptase inhibitors function through binding directly to the reverse transcriptase enzyme, thereby altering its conformation to prevent DNA binding. In some embodiments, the non-nucleoside reverse transcriptase inhibitor is etravirine. In some embodiments, the non-nucleoside reverse transcriptase inhibitor is rilpivirine.

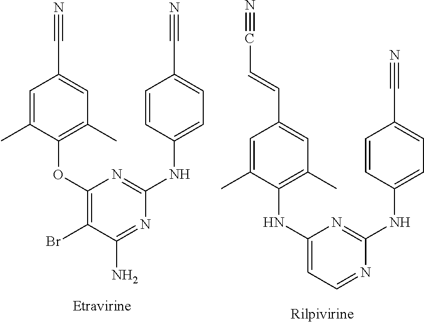 [SMILES: Cc1cc(C#N)cc(C)c1Oc1nc(Nc2ccc(C#N)cc2)nc(N)c1Br ; Cc1cc(/C=C/C#N)cc(C)c1Nc1ccnc(Nc2ccc(C#N)cc2)n1]

In some embodiments, the other antiviral agent is a protease inhibitor. Protease inhibitors function by competitively binding to HIV-1 protease. In some embodiments, the protease inhibitor is darunavir.

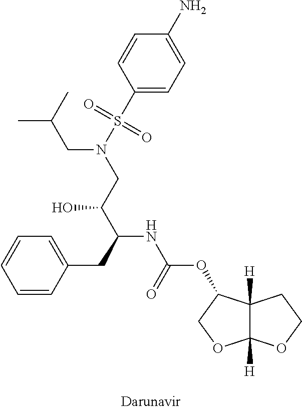 [SMILES: CC(C)CN(C[C@@H](O)[C@H](Cc1ccccc1)NC(=O)O[C@H]1CO[C@H]2OCC[C@H]21)S(=O)(=O)c1ccc(N)cc1]

In some embodiments, the other antiviral agent is an integrase inhibitor. Integrase inhibitors function through inhibition of the integrase enzyme, which interferes with the ability of HIV to incorporate its DNA into the host genome. In some embodiments, the integrase inhibitor is elvitegravir.

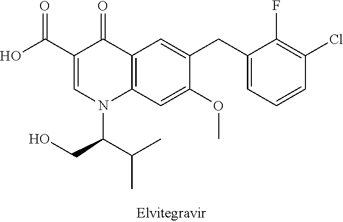 [SMILES: COc1cc2c(cc1Cc1cccc(Cl)c1F)c(=O)c(C(=O)O)cn2[C@H](CO)C(C)C]

In some embodiments, the other antiviral agent is a viral DNA polymerase inhibitor. Viral DNA polymerases function through inhibition of the viral DNA polymerase, which prevents viral genome replication and transcription. In some embodiments, the viral DNA polymerase inhibitor is ganciclovir. In some embodiments, the viral DNA polymerase is acyclovir. In some embodiments, the viral DNA polymerase is valganciclovir.

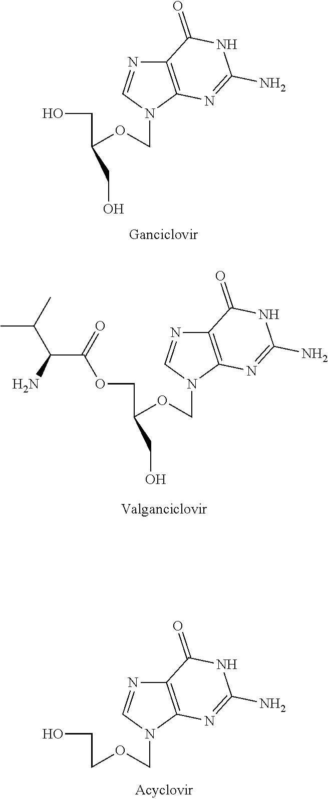

Exemplary Embodiments for Combination Therapy of Peramivir with Other Antiviral Agent

In some embodiments, there is provided a method of treating an infectious disease in an individual comprising administering to an individual: a) an effective amount of peramivir, and b) an effective amount of ganciclovir. In some embodiments, the ganciclovir is administered intravenously. In some embodiments, the ganciclovir is administered at about 5 mg/kg body weight. In some embodiments, the ganciclovir is administered at a constant rate over one hour. In some embodiments, the ganciclovir is administered every twelve hours. In some embodiments, the ganciclovir is administered for 12 to 14 days. In other embodiments, the ganciclovir is administered orally. In some embodiments, the ganciclovir is administered orally after at least 14-21 days of intravenous ganciclovir. In some embodiments, the individual is administered about 1000 mg ganciclovir three times daily. In some embodiments, the individual is administered about 500 mg ganciclovir six times daily. In other embodiments, the individual is administered about 500, 1000, 1500, 2000, or 2500 mg ganciclovir. In some embodiments, the individual is administered 3000-4000 mg ganciclovir or more. In some embodiments, the peramivir is administered intravenously. In other embodiments, the peramivir is administered intramuscularly. In other embodiments, the peramivir is administered orally. In some embodiments, the peramivir is administered as a one-time dose. In some embodiments, the peramivir is administered more than one dose. In some embodiments, the individual is administered about 600 mg peramivir. In some embodiments, the individual is administered 100, 200, 300, 400, 500, 600, 700, 800, 900, or 1000 mg peramivir. In some embodiments, the individual is administered 1500 or 2000 mg peramivir or more.

In some embodiments, there is provided a method of treating an infectious disease in an individual comprising administering to an individual: a) an effective amount of peramivir, and b) an effective amount of elvitegravir. In some embodiments the individual is administered elvitegravir orally. In some embodiments, the individual is administered about 85 mg elvitegravir. In other embodiments, the individual is administered about 150 mg elvitegravir. In other embodiments, the individual is administered about 12, 25, 50, 75, 100, 125, 150, 175, or 200 mg elvitegravir. In other embodiments, the individual is administered about 200, 300, 400, 500, 600, 700, 800, 900, or 1000 mg elvitegravir or more. In some embodiments, the peramivir is administered intravenously. In other embodiments, the peramivir is administered intramuscularly. In other embodiments, the peramivir is administered orally. In some embodiments, the peramivir is administered as a one-time dose. In some embodiments, the peramivir is administered more than one dose. In some embodiments, the individual is administered about 600 mg peramivir. In some embodiments, the individual is administered 100, 200, 300, 400, 500, 600, 700, 800, 900, or 1000 mg peramivir. In some embodiments, the individual is administered 1500 or 2000 mg peramivir or more.

In some embodiments, there is provided a method of treating an infectious disease in an individual comprising administering to an individual: a) an effective amount of peramivir, and b) an effective amount of valganciclovir. In some embodiments, the individual is administered valganciclovir orally. In some embodiments, the individual is administered about 450 mg valganciclovir twice daily. In some embodiments, the individual is administered valganciclovir for 21 days. In some embodiments, the individual is administered about 50, 100, 200, 300, 400, 500, 600, 700, 800, 900, or 1000 mg valganciclovir. In other embodiments, the individual is administered 1500-3000 mg valganciclovir or more. In some embodiments, the peramivir is administered intravenously. In other embodiments, the peramivir is administered intramuscularly. In other embodiments, the peramivir is administered orally. In some embodiments, the peramivir is administered as a one-time dose. In some embodiments, the peramivir is administered more than one dose. In some embodiments, the individual is administered about 600 mg peramivir. In some embodiments, the individual is administered 100, 200, 300, 400, 500, 600, 700, 800, 900, or 1000 mg peramivir. In some embodiments, the individual is administered 1500 or 2000 mg peramivir or more.

In some embodiments, there is provided a method of treating an infectious disease in an individual comprising administering to an individual: a) an effective amount of peramivir, and b) an effective amount of acyclovir. In some embodiments, the acyclovir is administered orally. In some embodiments, the individual is administered between about 200 and about 800 mg acyclovir. In some embodiments, the individual is administered about 100, 200, 300, 400, 500, 600, 700, 800, 900, or 1000 mg acyclovir. In some embodiments, the individual is administered 2000-5000 mg acyclovir or more. In some embodiments, the individual is administered acyclovir for 5 to 10 days. In other embodiments, the individual is administered acyclovir intravenously. In some embodiments, the individual is administered about 5 to about 10 mg/kg body weight acyclovir. In some embodiments, the individual is administered acyclovir every 8 hours. In some embodiments, the individual is administered acyclovir for about 5 to 10 days. In some embodiments, the peramivir is administered intravenously. In other embodiments, the peramivir is administered intramuscularly. In other embodiments, the peramivir is administered orally. In some embodiments, the peramivir is administered as a one-time dose. In some embodiments, the peramivir is administered more than one dose. In some embodiments, the individual is administered about 600 mg peramivir. In some embodiments, the individual is administered 100, 200, 300, 400, 500, 600, 700, 800, 900, or 1000 mg peramivir. In some embodiments, the individual is administered 1500 or 2000 mg peramivir or more.

In some embodiments, there is provided a method of treating an infectious disease in an individual comprising administering to an individual: a) an effective amount of peramivir, and b) an effective amount of abacavir. In some embodiments, the abacavir is administered orally. In some embodiments, the individual is administered about 600 mg daily. In some embodiments, the individual is administered about 600 mg daily in one dose. In other embodiments, the individual is administered about 300 mg twice daily. In some embodiments, the individual is administered about 100, 200, 300, 400, 500, 600, 700, 800, 900, or 1000 mg abacavir. In other embodiments, the individual is administered about 2000-5000 mg abacavir or more. In some embodiments, the peramivir is administered intravenously. In other embodiments, the peramivir is administered intramuscularly. In other embodiments, the peramivir is administered orally. In some embodiments, the peramivir is administered as a one-time dose. In some embodiments, the peramivir is administered more than one dose. In some embodiments, the individual is administered about 600 mg peramivir. In some embodiments, the individual is administered 100, 200, 300, 400, 500, 600, 700, 800, 900, or 1000 mg peramivir. In some embodiments, the individual is administered 1500 or 2000 mg peramivir or more.

In some embodiments, there is provided a method of treating an infectious disease in an individual comprising administering to an individual: a) an effective amount of peramivir, and b) an effective amount of darunavir. In some embodiments, the darunavir is administered orally. In some embodiments, the individual is administered about 800 mg daily. In other embodiments, the individual is administered about 400 mg twice daily. In other embodiments, the individual is administered about 100, 200, 300, 400, 500, 600, 700, 800, 900, or 1000 mg darunavir. In other embodiments, the individual is administered about 2000-5000 mg darunavir or more. In some embodiments, the peramivir is administered intravenously. In other embodiments, the peramivir is administered intramuscularly. In other embodiments, the peramivir is administered orally. In some embodiments, the peramivir is administered as a one-time dose. In some embodiments, the peramivir is administered more than one dose. In some embodiments, the individual is administered about 600 mg peramivir. In some embodiments, the individual is administered 100, 200, 300, 400, 500, 600, 700, 800, 900, or 1000 mg peramivir. In some embodiments, the individual is administered 1500 or 2000 mg peramivir or more.

In some embodiments, there is provided a method of treating an infectious disease in an individual comprising administering to an individual: a) an effective amount of peramivir, and b) an effective amount of etravirine. In some embodiments, the etravirine is administered orally. In some embodiments, the individual is administered about 200 mg daily. In some embodiments, the individual is administered about 100 mg twice daily. In some embodiments, the individual is administered about 100, 200, 300, 400, 500, 600, 700, 800, 900, or 1000 mg etravirine. In other embodiments, the individual is administered about 2000-5000 mg etravirine or more. In some embodiments, the peramivir is administered intravenously. In other embodiments, the peramivir is administered intramuscularly. In other embodiments, the peramivir is administered orally. In some embodiments, the peramivir is administered as a one-time dose. In some embodiments, the peramivir is administered more than one dose. In some embodiments, the individual is administered about 600 mg peramivir. In some embodiments, the individual is administered 100, 200, 300, 400, 500, 600, 700, 800, 900, or 1000 mg peramivir. In some embodiments, the individual is administered 1500 or 2000 mg peramivir or more.

In some embodiments, there is provided a method of treating an infectious disease in an individual comprising administering to an individual: a) an effective amount of peramivir, and b) an effective amount of rilpivirine. In some embodiments, the rilpivirine is administered orally. In some embodiments, the individual is administered about 25 mg daily. In some embodiments, the individual is administered about 10, 20, 30, 40, 50, 60, 70, 80, 90, or 100 mg rilpivirine. In other embodiments, the individual is administered about 150 to about 500 mg rilpivirine. In other embodiments, the rilpivirine is administered intramuscularly. In some embodiments, the rilpivirine is administered intramuscularly as an extended-release injectable suspension. In some embodiments, the individual is administered about 300 mg/mL rilpivirine. In some embodiments, the individual is administered rilpivirine intramuscularly once monthly. In some embodiments, the individual is administered rilpivirine once monthly only after oral introduction of rilpivirine. In some embodiments, the peramivir is administered intravenously. In other embodiments, the peramivir is administered intramuscularly. In other embodiments, the peramivir is administered orally. In some embodiments, the peramivir is administered as a one-time dose. In some embodiments, the peramivir is administered more than one dose. In some embodiments, the individual is administered about 600 mg peramivir. In some embodiments, the individual is administered 100, 200, 300, 400, 500, 600, 700, 800, 900, or 1000 mg peramivir. In some embodiments, the individual is administered 1500 or 2000 mg peramivir or more.

In some embodiments, there is provided a method of treating an infectious disease in an individual comprising administering to an individual: a) an effective amount of peramivir, and b) an effective amount of stavudine. In some embodiments, the stavudine is administered orally. In some embodiments, the individual is administered less than 30 mg/60 kg body weight. In some embodiments, the individual is administered stavudine every 12 hours. In some embodiments, the individual is administered at least 40 mg/60 kg body weight. In some embodiments, the individual is administered stavudine every 12 hours. In some embodiments, the individual is administered about 5, 10, 15, 20, or 25 mg stavudine per 60 kg body weight. In other embodiments, the individual is administered about 35 mg stavudine per 60 kg body weight. In other embodiments, the individual is administered about 45, 50, 55, 60, 65, 70, 75, 80, 85, 90, 95, or 100 mg stavudine per 60 kg body weight. In other embodiments, the individual is administered about 150 to about 500 mg stavudine per kg body weight. In some embodiments, the peramivir is administered intravenously. In other embodiments, the peramivir is administered intramuscularly. In other embodiments, the peramivir is administered orally. In some embodiments, the peramivir is administered as a one-time dose. In some embodiments, the peramivir is administered more than one dose. In some embodiments, the individual is administered about 600 mg peramivir. In some embodiments, the individual is administered 100, 200, 300, 400, 500, 600, 700, 800, 900, or 1000 mg peramivir. In some embodiments, the individual is administered 1500 or 2000 mg peramivir or more.

In some embodiments, there is provided a method of treating an infectious disease in an individual comprising administering to an individual: a) an effective amount of peramivir, and b) an effective amount of didanosine. In some embodiments, the didanosine is administered orally. In some embodiments, the individual weighs at least about 60 kg. In some embodiments, the individual is administered at least about 200 mg didanosine twice daily. In some embodiments, the individual weighs less than about 60 kg. In some embodiments, the individual is administered about 125 mg didanosine twice daily. In some embodiments, the individual is administered about 25, 75, 125, 175, 225, 275, 325, 375, 425, 475, or 500 mg didanosine. In other embodiments, the individual is administered about 60 to about 2000 mg didanosine or more. In some embodiments, the peramivir is administered intravenously. In other embodiments, the peramivir is administered intramuscularly. In other embodiments, the peramivir is administered orally. In some embodiments, the peramivir is administered as a one-time dose. In some embodiments, the peramivir is administered more than one dose. In some embodiments, the individual is administered about 600 mg peramivir. In some embodiments, the individual is administered 100, 200, 300, 400, 500, 600, 700, 800, 900, or 1000 mg peramivir. In some embodiments, the individual is administered 1500 or 2000 mg peramivir or more.

In some embodiments, there is a pharmaceutical composition comprising peramivir and at least one other antiviral agent. In some embodiments, the other antiviral agent is a reverse transcriptase inhibitor. In some embodiments, the reverse transcriptase inhibitor is a nucleoside analog reverse transcriptase inhibitor. In some embodiments, the nucleoside analog reverse transcriptase inhibitor is abacavir. In other embodiments, the nucleoside analog reverse transcriptase inhibitor is didanosine. In other embodiments, the nucleoside analog reverse transcriptase inhibitor is stavudine.

In some embodiments, the nucleoside analog reverse transcriptase inhibitor is a thymidine analogue. In some embodiments, the thymidine analogue is stavudine. In some embodiments, the nucleoside analogue reverse transcriptase inhibitor is a cytidine analogue. In some embodiments, the nucleoside analogue reverse transcriptase inhibitor is a guanosine analogue. In some embodiments, the guanosine analogue is abacavir. In some embodiments, the nucleoside analog reverse transcriptase inhibitor is an adenosine analogue. In some embodiments, the adenosine analogue is didanosine.

In some embodiments, the reverse transcriptase inhibitor is a non-nucleoside reverse transcriptase inhibitor. Non-nucleoside reverse transcriptase inhibitors function through binding directly to the reverse transcriptase enzyme, thereby altering its conformation to prevent DNA binding. In some embodiments, the non-nucleoside reverse transcriptase inhibitor is etravirine. In some embodiments, the non-nucleoside reverse transcriptase inhibitor is rilpivirine.

In some embodiments, the other antiviral agent is a protease inhibitor. Protease inhibitors function by competitively binding to HIV-1 protease. In some embodiments, the protease inhibitor is darunavir.

In some embodiments, the other antiviral agent is an integrase inhibitor. Integrase inhibitors function through inhibition of the integrase enzyme, which interferes with the ability of HIV to incorporate its DNA into the host genome. In some embodiments, the integrase inhibitor is elvitegravir.

In some embodiments, the other antiviral agent is a viral DNA polymerase inhibitor. Viral DNA polymerases function through inhibition of the viral DNA polymerase, which prevents viral genome replication and transcription. In some embodiments, the viral DNA polymerase inhibitor is ganciclovir. In some embodiments, the viral DNA polymerase is acyclovir. In some embodiments, the viral DNA polymerase is valganciclovir.

Pharmaceutical Compositions

Also provided are pharmaceutical compositions comprising peramivir and other antiviral agents. Pharmaceutical compositions containing the compounds of the present disclosure may be in any form suitable for the intended method of administration. In some embodiments, the pharmaceutical composition is suitable for oral administration. Formulations suitable for oral administration can consist of (a) liquid solutions, such as an effective amount of the compound dissolved in diluents, such as water, saline, or orange juice, (b) capsules, sachets or tablets, each containing a predetermined amount of the active ingredient, as solids or granules, (c) suspensions in an appropriate liquid, and (d) suitable emulsions. Tablet forms can include one or more of lactose, mannitol, corn starch, potato starch, microcrystalline cellulose, acacia, gelatin, colloidal silicon dioxide, croscarmellose sodium, talc, magnesium stearate, stearic acid, and other excipients, colorants, diluents, buffering agents, moistening agents, preservatives, flavoring agents, and pharmacologically compatible excipients. Lozenge forms can comprise the active ingredient in a flavor, usually sucrose and acacia or tragacanth, as well as pastilles comprising the active ingredient in an inert base, such as gelatin and glycerin, or sucrose and acacia, emulsions, gels, and the like containing, in addition to the active ingredient, such excipients as are known in the art.

Examples of suitable carriers, excipients, and diluents include, but are not limited to, lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, saline solution, syrup, methylcellulose, methyl- and propylhydroxybenzoates, talc, magnesium stearate, and mineral oil. The formulations can additionally include lubricating agents, wetting agents, emulsifying and suspending agents, preserving agents, sweetening agents or flavoring agents.

Solid dosage forms for oral administration may include capsules, tablets, pills, powders and granules. In such solid dosage forms, the active compound may be admixed with at least one inert diluent such as sucrose, lactose, or starch. Such dosage forms may also comprise additional substances other than inert diluents, e.g., lubricating agents such as magnesium stearate. In the case of capsules, tablets, and pills, the dosage forms may also comprise buffering agents. Tablets and pills can additionally be prepared with enteric coatings.

Liquid dosage forms for oral administration may include pharmaceutically acceptable emulsions, solutions, suspensions, syrups, and elixirs containing inert diluents commonly used in the art, such as water. Such compositions may also comprise adjuvants, such as wetting agents, emulsifying and suspending agents, cyclodextrins, and sweetening, flavoring, and perfuming agents.

In some embodiments, the formulation is suitable for intravenous administration.

In other embodiments, the formulation is suitable for intramuscular administration. Formulations suitable for intravenous and intramuscular administration include aqueous and non-aqueous, isotonic sterile injection solutions, which can contain anti-oxidants, buffers, bacteriostats, and solutes that render the formulation compatible with the blood of the intended recipient, and aqueous and non-aqueous sterile suspensions that can include suspending agents, solubilizers, thickening agents, stabilizers, and preservatives. The formulations can be presented in unit-dose or multi-dose sealed containers, such as ampules and vials, and can be stored in a freeze-dried (lyophilized) condition requiring only the addition of the sterile liquid excipient, for example, water, for injections, immediately prior to use. Extemporaneous injection solutions and suspensions can be prepared from sterile powders, granules, and tablets of the kind previously described.

In some embodiments, the ratio by weight of the peramivir and the other antiviral agent in the pharmaceutical composition is about 1 to 1. In some embodiments, the weight ratio may be between about 0.001 to about 1 and about 1000 to about 1, or between about 0.01 to about 1 and 100 to about 1. In some embodiments, the ratio by weight of the peramivir and the other antiviral is less than any of about 1000:1, 900:1, 800:1, 700:1, 600:1, 500:1, 400:1, 300:1, 200:1, 100:1, 50:1, 30:1, 10:1, 9:1, 8:1, 7:1, 6:1, 5:1, 4:1, 3:1, 2:1, and 1:1. In some embodiments, the ratio by weight of the peramivir and the other antiviral is more than any of about 1:1, 2:1, 3:1, 4:1, 5:1, 6:1, 7:1, 8:1, 9:1, 10:1, 30:1, 50:1, 75:1, 100:1, 200:1, 300:1, 400:1, 500:1, 600:1, 700:1, 800:1, 900:1, and 1000:1. In some embodiments, the ratio by weight of the peramivir and the other antiviral is less than any of about 1:1, 1:2, 1:3, 1:4, 1:5, 1:6, 1:7, 1:8, 1:9, 1:10, 1:30, 1:50, 1:100, 1:200, 1:300, 1:400, 1:500, 1:600, 1:700, 1:800, 1:900, and 1:1000. In other embodiments, the ratio by weight of the peramivir and the other antiviral is more than any of about 1:1000, 1:900, 1:800, 1:700, 1:600, 1:500, 1:400, 1:300, 1:200, 1:100, 1:50, 1:30, 1:10, 1:9, 1:8, 1:7, 1:6, 1:5, 1:4, 1:3, 1:2, and 1:1. Other ratios are contemplated.

In some embodiments, there is provided a pharmaceutical composition comprising: a) peramivir, and b) ganciclovir. In some embodiments, the composition is suitable for intravenous administration. In other embodiments, the composition is suitable for oral administration. In other embodiments, the composition is suitable for intramuscular administration. In some embodiments, the weight ratio of the peramivir to ganciclovir in the pharmaceutical composition is about any of 10:1, 9:2, 8:1, 7:1, 6:1, 5:1. 4:1, 3:1, 2:1, 1:1, 1:2, 1:3, 1:4, 1:5, 1:6, 1:7, 1:8, 1:9, or 1:10.

In some embodiments, there is provided a pharmaceutical composition comprising: a) peramivir, and b) elvitegravir. In some embodiments, the composition is suitable for intravenous administration. In other embodiments, the composition is suitable for oral administration. In other embodiments, the composition is suitable for intramuscular administration. In some embodiments, the weight ratio of the peramivir to elvitegravir in the pharmaceutical composition is about any of 10:1, 9:2, 8:1, 7:1, 6:1, 5:1. 4:1, 3:1, 2:1, 1:1, 1:2, 1:3, 1:4, 1:5, 1:6, 1:7, 1:8, 1:9, or 1:10.

In some embodiments, there is provided a pharmaceutical composition comprising: a) peramivir, and b) valganciclovir. In some embodiments, the composition is suitable for intravenous administration. In other embodiments, the composition is suitable for oral administration. In other embodiments, the composition is suitable for intramuscular administration. In some embodiments, the weight ratio of the peramivir to valganciclovir in the pharmaceutical composition is about any of 10:1, 9:2, 8:1, 7:1, 6:1, 5:1. 4:1, 3:1, 2:1, 1:1, 1:2, 1:3, 1:4, 1:5, 1:6, 1:7, 1:8, 1:9, or 1:10.

In some embodiments, there is provided a pharmaceutical composition comprising: a) peramivir, and b) acyclovir. In some embodiments, the composition is suitable for intravenous administration. In other embodiments, the composition is suitable for oral administration. In other embodiments, the composition is suitable for intramuscular administration. In some embodiments, the weight ratio of the peramivir to acyclovir in the pharmaceutical composition is about any of 10:1, 9:2, 8:1, 7:1, 6:1, 5:1. 4:1, 3:1, 2:1, 1:1, 1:2, 1:3, 1:4, 1:5, 1:6, 1:7, 1:8, 1:9, or 1:10.

In some embodiments, there is provided a pharmaceutical composition comprising: a) peramivir, and b) abacavir. In some embodiments, the composition is suitable for intravenous administration. In other embodiments, the composition is suitable for oral administration. In other embodiments, the composition is suitable for intramuscular administration. In some embodiments, the weight ratio of the peramivir to abacavir in the pharmaceutical composition is about any of 10:1, 9:2, 8:1, 7:1, 6:1, 5:1. 4:1, 3:1, 2:1, 1:1, 1:2, 1:3, 1:4, 1:5, 1:6, 1:7, 1:8, 1:9, or 1:10.

In some embodiments, there is provided a pharmaceutical composition comprising: a) peramivir, and b) darunavir. In some embodiments, the composition is suitable for intravenous administration. In other embodiments, the composition is suitable for oral administration. In other embodiments, the composition is suitable for intramuscular administration. In some embodiments, the weight ratio of the peramivir to darunavir in the pharmaceutical composition is about any of 10:1, 9:2, 8:1, 7:1, 6:1, 5:1. 4:1, 3:1, 2:1, 1:1, 1:2, 1:3, 1:4, 1:5, 1:6, 1:7, 1:8, 1:9, or 1:10.

In some embodiments, there is provided a pharmaceutical composition comprising: a) peramivir, and b) etravirine. In some embodiments, the composition is suitable for intravenous administration. In other embodiments, the composition is suitable for oral administration. In other embodiments, the composition is suitable for intramuscular administration. In some embodiments, the weight ratio of the peramivir to etravirine in the pharmaceutical composition is about any of 10:1, 9:2, 8:1, 7:1, 6:1, 5:1. 4:1, 3:1, 2:1, 1:1, 1:2, 1:3, 1:4, 1:5, 1:6, 1:7, 1:8, 1:9, or 1:10.

In some embodiments, there is provided a pharmaceutical composition comprising: a) peramivir, and b) rilpivirine. In some embodiments, the composition is suitable for intravenous administration. In other embodiments, the composition is suitable for oral administration. In other embodiments, the composition is suitable for intramuscular administration. In some embodiments, the weight ratio of the peramivir to rilpivirine in the pharmaceutical composition is about any of 10:1, 9:2, 8:1, 7:1, 6:1, 5:1. 4:1, 3:1, 2:1, 1:1, 1:2, 1:3, 1:4, 1:5, 1:6, 1:7, 1:8, 1:9, or 1:10.

In some embodiments, there is provided a pharmaceutical composition comprising: a) peramivir, and b) stavudine. In some embodiments, the composition is suitable for intravenous administration. In other embodiments, the composition is suitable for oral administration. In other embodiments, the composition is suitable for intramuscular administration. In some embodiments, the weight ratio of the peramivir to stavudine in the pharmaceutical composition is about any of 10:1, 9:2, 8:1, 7:1, 6:1, 5:1. 4:1, 3:1, 2:1, 1:1, 1:2, 1:3, 1:4, 1:5, 1:6, 1:7, 1:8, 1:9, or 1:10.

In some embodiments, there is provided a pharmaceutical composition comprising: a) peramivir, and b) didanosine. In some embodiments, the composition is suitable for intravenous administration. In other embodiments, the composition is suitable for oral administration. In other embodiments, the composition is suitable for intramuscular administration. In some embodiments, the weight ratio of the peramivir to didanosine in the pharmaceutical composition is about any of 10:1, 9:2, 8:1, 7:1, 6:1, 5:1. 4:1, 3:1, 2:1, 1:1, 1:2, 1:3, 1:4, 1:5, 1:6, 1:7, 1:8, 1:9, or 1:10.

Also within the scope of the present disclosure is a kit comprising a combination therapy described herein. The kit can include one or more other elements including: instructions for use; devices or other materials for preparing the compositions for administration; pharmaceutically acceptable carriers; and devices or other materials for administration to a subject.

The combination therapies disclosed herein have in vitro and in vivo therapeutic and/or prophylactic utilities. For example, the compositions disclosed herein can be administered to cells in culture, in vitro or ex vivo, or to a subject, e.g., a human subject, to treat, prevent, and/or diagnose a variety of disorders, such as viral infection, e.g., SARS-CoV-2 infection.

Treatment with Cidofovir

Methods

Provided herein are methods for treating and/or preventing coronavirus-associated infection in an individual comprising administering to the individual an effective amount of cidofovir and a second antiviral agent. In some embodiments, the second antiviral agent is a reverse transcriptase inhibitor. In other embodiments, the other antiviral agent is a viral DNA polymerase inhibitor.

Certain aspects of the present disclosure relate to viral infections. In some embodiments, the virus is an enveloped virus. Examples of enveloped viruses are well known in the art and include, without limitation, the virus families of Arenavirus, Arterivirus, Asfarvirus, Baculovirus, Bunyavirus, Coronavirus, Cystovirus, Deltavirus, Filovirus, Flavivirus, Fusellovirus, Hepadnavirus, Herpesvirus, Iridovirus, Lipothrixivirus, Orthomyxovirus, Paramyxovirus, Plasmavirus, Polydnavirus, Poxvirus, Retrovirus, Rhabdovirus, and Togavirus. In some embodiments, the virus is a Coronavirus, e.g., severe acute respiratory syndrome coronavirus 1 (SARS-CoV-1), severe acute respiratory syndrome coronavirus 2 (SARS-CoV-2), or Middle East respiratory syndrome-related coronavirus (MERS-CoV). In some embodiments, the virus is an Orthomyxovirus, e.g., influenza virus A, B, or C. In some embodiments, the virus is an Orthopneumovirus, e.g., respiratory syncytial virus (RSV).

In some embodiments, an individual has been diagnosed with a coronavirus-associated disease. In some embodiments, an individual has been diagnosed with Severe Acute Respiratory Syndrome Coronavirus 2 (COVID-19). In some embodiments, an individual has or has been diagnosed with acute respiratory distress syndrome (ARDS), e.g., prior to treatment with the combination therapy. Methods for diagnosis of ARDS include, without limitation, chest X-ray, CT scanning, and/or measurement of oxygen levels. In some embodiments, an individual has been diagnosed with an Angiotensin-Converting Enzyme 2 (ACE2)-associated disease. In some embodiments, an individual has been diagnosed with Severe Acute Respiratory Syndrome (SARS). In some embodiments, an individual has been diagnosed with Middle East Respiratory Syndrome (MERS).

In some embodiments, an individual is treated with a combination of cidofovir and another antiviral agent. In some embodiments, the other antiviral agent is a reverse transcriptase inhibitor. In some embodiments, the reverse transcriptase inhibitor is a nucleoside analog reverse transcriptase inhibitor. In some embodiments the nucleoside analog reverse transcriptase inhibitor is zidovudine. In some embodiments, the nucleoside analog reverse transcriptase inhibitor is stavudine.

In some embodiments, the other antiviral agent is a DNA polymerase inhibitor. In some embodiments, the DNA polymerase inhibitor is valacyclovir.

In some embodiments, the other antiviral agent is administered orally. In some embodiments, the other antiviral agent is administered intramuscularly. In other embodiments, the other antiviral agent is administered intravenously. In some embodiments, the cidofovir and the other antiviral agent are administered simultaneously.

In some embodiments, the cidofovir and other antiviral agent are administered in a single composition. In some embodiments, the single composition is administered orally. In some embodiments, the single composition is administered intravenously. In some embodiments, the single composition is administered intramuscularly.

The composition comprising cidofovir and the other antiviral agent can be administered simultaneously (i.e., simultaneous administration and/or sequentially (i.e., sequential administration)).

In some embodiments, the cidofovir and the other antiviral agent are administered simultaneously. The term “simultaneous administration,” as used herein, means that the cidofovir and the other antiviral agent are administered with a time separation no more than about 15 minutes(s), such as no more than about any of 10, 5, or 1 minutes. When the drugs are administered simultaneously, the cidofovir and the other antiviral compound may be contained in the same composition (e.g., a composition comprising the cidofovir and the other antiviral agent) or in separate compositions (e.g., the cidofovir is contained in one composition and the other antiviral agent is contained in another composition).

In some embodiments, the cidofovir and the other antiviral agent are administered sequentially. The term “sequential administration” as used herein means that the cidofovir and the other antiviral agent are administered with a time separation of more than about 15 minutes, such as more than any of 20, 30, 40, 50, or more minutes. Either the cidofovir or the other antiviral agent may be administered first. In some embodiments, the cidofovir is administered prior to administration of the other antiviral agent. In some embodiments, the cidofovir is administered following administration of the other antiviral agent. The cidofovir and the other antiviral agent are contained in separate compositions, which may be contained in the same or different packages.

In some embodiments, the individual is a human. In some embodiments, the individual suffers from a pre-existing health condition correlated with poor prognosis following SARS-CoV2-disease. In some embodiments, the pre-existing health condition is cancer. In some embodiments, the pre-existing health condition is chronic kidney disease. In some embodiments, the pre-existing health condition is chronic obstructive pulmonary disease. In some embodiments, the pre-existing health condition is Down Syndrome. In some embodiments, the pre-existing health condition is a heart condition. In some embodiments, the pre-existing health condition is heart failure. In some embodiments, the pre-existing health condition is coronary artery disease. In some embodiments, the pre-existing health condition is cardiomyopathy. In some embodiments, the pre-existing health condition is an immunocompromised state. In some embodiments, the pre-existing health condition is obesity. In some embodiments, the pre-existing health condition is pregnancy. In some embodiments, the pre-existing health condition is sickle cell disease. In some embodiments, the pre-existing health condition is smoking. In some embodiments, the pre-existing health condition is Type I diabetes mellitus. In some embodiments, the pre-existing health condition is Type 2 diabetes mellitus. In some embodiments, the pre-existing health condition is asthma. In some embodiments, the pre-existing health condition is cerebrovascular disease. In some embodiments, the pre-existing health condition is cystic fibrosis. In some embodiments, the pre-existing health condition is hypertension. In some embodiments, the pre-existing health condition is a neurologic condition. In some embodiments, the pre-existing health condition is liver disease. In some embodiments, the pre-existing health condition is pulmonary fibrosis. In some embodiments, the pre-existing health condition is thalassemia. In some embodiments, the pre-existing health condition is 65 years or greater of age.

In some embodiments, the cidofovir and other antiviral is accompanied by a second therapy administered to the individual. In some embodiments, the second therapy is remdesivir. In some embodiments, the second therapy is monoclonal antibody. In some embodiments, the monoclonal antibody is targeted against SARS-CoV-2. In some embodiments, the second therapy is casirivimab and imdevimab. In some embodiments, the second therapy is administered intravenously. In some embodiments, the second therapy is mechanical ventilation.

Cidofovir

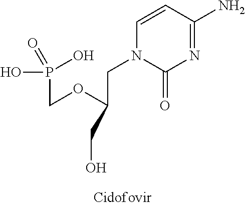 [SMILES: Nc1ccn(C[C@@H](CO)OCP(=O)(O)O)c(=O)n1]

Cidofovir is an antiviral compound that belongs to the class of nucleoside analogues. Nucleoside analogs are first converted into an active compound through intracellular phosphorylation. The phosphorylated form competes with cellular nucleotides and inhibits viral replication enzymes by introducing a chain terminator into the growing DNA strand during transcription.

Cidofovir is injectable and has been approved for the treatment of cytomegalovirus (CMV) retinitis in people with AIDS. In some embodiments, the cidofovir is administered intravenously. In other embodiments, the cidofovir is administered intramuscularly. In other embodiments, the cidofovir is administered orally. In some embodiments, the individual is administered a one-time dose. In some embodiments, the individual is administered more than one dose. In some embodiments, the individual is administered about 5 mg per kg body weight cidofovir. In some embodiments, the cidofovir is administered at a constant rate over one hour. In some embodiments, the cidofovir is administered once per week for two weeks. In some embodiments, the individual is administered 300 mg cidofovir. In other embodiments, the individual is administered about 25, 50, 100, 150, 200, 250, 300, 350, 400, 450, 500, 550, 600, 650, 700, 750, 800, 850, 900, 950, or 1000 mg cidofovir. In some embodiments the individual is administered about 1500-2500 mg cidofovir or more.

Reverse Transcriptase Inhibitors

Reverse transcriptase inhibitors are a class of anti-viral drug. In particular, the class is used to treat HIV/AIDS. Reverse transcriptase inhibitors inhibit the activity of reverse transcriptase, an enzyme required for the replication of retroviruses.

Suitable reverse transcriptase inhibitors described herein include, for example, 1) nucleoside analog reverse transcriptase inhibitors (NARTIs or NRTIs); 2) non-nucleoside reverse-transcriptase inhibitors (NNRTIs); 3) nucleoside reverse transcriptase translocation inhibitors (NNRTIs); and 4) Portmanteau inhibitors.

In some embodiments, the other antiviral agent is a reverse transcriptase inhibitor. In some embodiments, the reverse transcriptase inhibitor is a nucleoside analog reverse transcriptase inhibitor. Nucleoside analog reverse transcriptase inhibitors are first converted into an active compound through intracellular phosphorylation to a triphosphate form. The triphosphate form of the nucleoside analog inhibitor competes with cellular nucleotides and inhibits the reverse transcriptase enzyme by introducing a chain terminator into the growing DNA strand during reverse transcription. In some embodiments, the other antiviral is a nucleoside analog reverse transcriptase inhibitor. In some embodiments, the nucleoside analog reverse transcriptase inhibitor is zidovudine. In some embodiments, the nucleoside reverse transcriptase inhibitor is stavudine.

In some embodiments, the nucleoside analog reverse transcriptase inhibitor is a thymidine analogue. In some embodiments, the thymidine analogue is zidovudine. In some embodiments, the thymidine analogue is stavudine. In some embodiments, the nucleoside analogue reverse transcriptase inhibitor is a cytidine analogue. In some embodiments, the nucleoside analogue reverse transcriptase inhibitor is a guanosine analogue. In some embodiments, the nucleoside analog reverse transcriptase inhibitor is an adenosine analogue.

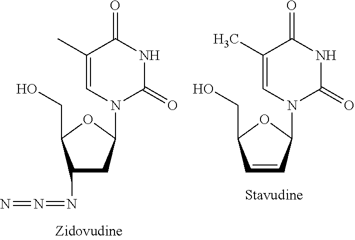

In some embodiments, the other antiviral agent is a viral DNA polymerase inhibitor. Viral DNA polymerases function through inhibition of the viral DNA polymerase, which prevents viral genome replication and transcription. In some embodiments, the viral DNA polymerase inhibitor is valacyclovir.

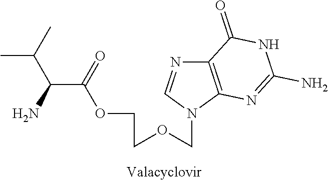 [SMILES: CC(C)[C@H](N)C(=O)OCCOCn1cnc2c(=O)[nH]c(N)nc21]

Exemplary Embodiments for Combination Therapy of Cidofovir with Other Antiviral Agent

In some embodiments, there is provided a method of treating an infectious disease in an individual comprising administering to an individual: a) an effective amount of cidofovir, and b) an effective amount of zidovudine. In some embodiments, the individual is administered zidovudine orally. In some embodiments, the individual is administered about 600 mg zidovudine once daily. In some embodiments, the individual is administered about 100, 200, 300, 400, 500, 600, 700, 800, 900, or 1000 mg zidovudine. In other embodiments, the individual is administered about 1000-2000 mg zidovudine or more. In some embodiments, the cidofovir is administered intravenously. In other embodiments, the cidofovir is administered intramuscularly. In other embodiments, the cidofovir is administered orally. In some embodiments, the individual is administered a one-time dose. In some embodiments, the individual is administered more than one dose. In some embodiments, the individual is administered about 5 mg per kg body weight cidofovir. In some embodiments, the cidofovir is administered at a constant rate over one hour. In some embodiments, the cidofovir is administered once per week for two weeks. In some embodiments, the individual is administered 300 mg cidofovir. In other embodiments, the individual is administered about 25, 50, 100, 150, 200, 250, 300, 350, 400, 450, 500, 550, 600, 650, 700, 750, 800, 850, 900, 950, or 1000 mg cidofovir. In some embodiments the individual is administered about 1500-2500 mg cidofovir or more.

In some embodiments, there is provided a method of treating an infectious disease in an individual comprising administering to an individual: a) an effective amount of cidofovir, and b) an effective amount of stavudine. In some embodiments, the stavudine is administered orally. In some embodiments, the individual is administered less than 30 mg/60 kg body weight. In some embodiments, the individual is administered stavudine every 12 hours. In some embodiments, the individual is administered at least 40 mg/60 kg body weight. In some embodiments, the individual is administered stavudine every 12 hours. In some embodiments, the individual is administered about 5, 10, 15, 20, or 25 mg stavudine per 60 kg body weight. In other embodiments, the individual is administered about 35 mg stavudine per 60 kg body weight. In other embodiments, the individual is administered about 45, 50, 55, 60, 65, 70, 75, 80, 85, 90, 95, or 100 mg stavudine per 60 kg body weight. In other embodiments, the individual is administered about 150 to about 500 mg stavudine per kg body weight. In some embodiments, the individual is administered less than about 20-40 mg stavudine. In other embodiments, the individual is administered more than about 40-60 mg stavudine. In some embodiments, the cidofovir is administered intravenously. In other embodiments, the cidofovir is administered intramuscularly. In other embodiments, the cidofovir is administered orally. In some embodiments, the individual is administered a one-time dose. In some embodiments, the individual is administered more than one dose. In some embodiments, the individual is administered about 5 mg per kg body weight cidofovir. In some embodiments, the cidofovir is administered at a constant rate over one hour. In some embodiments, the cidofovir is administered once per week for two weeks. In some embodiments, the individual is administered 300 mg cidofovir. In other embodiments, the individual is administered about 25, 50, 100, 150, 200, 250, 300, 350, 400, 450, 500, 550, 600, 650, 700, 750, 800, 850, 900, 950, or 1000 mg cidofovir. In some embodiments the individual is administered about 1500-2500 mg cidofovir or more.

In some embodiments, there is provided a method of treating an infectious disease in an individual comprising administering to an individual: a) an effective amount of cidofovir, and b) an effective amount of valacyclovir. In some embodiments, the valacyclovir is administered orally. In some embodiments, the valacyclovir is administered 1 gram twice daily for 10 days. In some embodiments, the individual is administered about 0.25, 0.5, 0.75, 1, 2, 3, 4, or 5 grams valacyclovir. In other embodiments, the individual is administered 5-8 grams valacyclovir or more. In some embodiments, the cidofovir is administered intravenously. In other embodiments, the cidofovir is administered intramuscularly. In other embodiments, the cidofovir is administered orally. In some embodiments, the individual is administered a one-time dose. In some embodiments, the individual is administered more than one dose. In some embodiments, the individual is administered about 5 mg per kg body weight cidofovir. In some embodiments, the cidofovir is administered at a constant rate over one hour. In some embodiments, the cidofovir is administered once per week for two weeks. In some embodiments, the individual is administered 300 mg cidofovir. In other embodiments, the individual is administered about 25, 50, 100, 150, 200, 250, 300, 350, 400, 450, 500, 550, 600, 650, 700, 750, 800, 850, 900, 950, or 1000 mg cidofovir. In some embodiments the individual is administered about 1500-2500 mg cidofovir or more.

In some embodiments, there is a pharmaceutical composition comprising cidofovir and at least one other antiviral agent. In some embodiments, the other antiviral agent is a reverse transcriptase inhibitor. In some embodiments, the reverse transcriptase inhibitor is a nucleoside analog reverse transcriptase inhibitor. In some embodiments, the nucleoside analog reverse transcriptase inhibitor is zidovudine. In other embodiments, the nucleoside analog reverse transcriptase inhibitor is stavudine.

In some embodiments, the nucleoside analog reverse transcriptase inhibitor is a thymidine analogue. In some embodiments, the thymidine analogue is stavudine. In other embodiments, the thymidine analogue is zidovudine. In some embodiments, the nucleoside analogue reverse transcriptase inhibitor is a cytidine analogue. In some embodiments, the nucleoside analogue reverse transcriptase inhibitor is a guanosine analogue. In some embodiments, the nucleoside analog reverse transcriptase inhibitor is an adenosine analogue.

In some embodiments, the other antiviral agent is a viral DNA polymerase inhibitor. Viral DNA polymerases function through inhibition of the viral DNA polymerase, which prevents viral genome replication and transcription. In some embodiments, the viral DNA polymerase inhibitor is valacyclovir.

Pharmaceutical Compositions

Also provided are pharmaceutical compositions comprising cidofovir and other antiviral agents. Pharmaceutical compositions containing the compounds of the present disclosure may be in any form suitable for the intended method of administration. In some embodiments, the pharmaceutical composition is suitable for oral administration. Formulations suitable for oral administration can consist of (a) liquid solutions, such as an effective amount of the compound dissolved in diluents, such as water, saline, or orange juice, (b) capsules, sachets or tablets, each containing a predetermined amount of the active ingredient, as solids or granules, (c) suspensions in an appropriate liquid, and (d) suitable emulsions. Tablet forms can include one or more of lactose, mannitol, corn starch, potato starch, microcrystalline cellulose, acacia, gelatin, colloidal silicon dioxide, croscarmellose sodium, talc, magnesium stearate, stearic acid, and other excipients, colorants, diluents, buffering agents, moistening agents, preservatives, flavoring agents, and pharmacologically compatible excipients. Lozenge forms can comprise the active ingredient in a flavor, usually sucrose and acacia or tragacanth, as well as pastilles comprising the active ingredient in an inert base, such as gelatin and glycerin, or sucrose and acacia, emulsions, gels, and the like containing, in addition to the active ingredient, such excipients as are known in the art.

Examples of suitable carriers, excipients, and diluents include, but are not limited to, lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, saline solution, syrup, methylcellulose, methyl- and propylhydroxybenzoates, talc, magnesium stearate, and mineral oil. The formulations can additionally include lubricating agents, wetting agents, emulsifying and suspending agents, preserving agents, sweetening agents or flavoring agents.

Solid dosage forms for oral administration may include capsules, tablets, pills, powders and granules. In such solid dosage forms, the active compound may be admixed with at least one inert diluent such as sucrose, lactose, or starch. Such dosage forms may also comprise additional substances other than inert diluents, e.g., lubricating agents such as magnesium stearate. In the case of capsules, tablets, and pills, the dosage forms may also comprise buffering agents. Tablets and pills can additionally be prepared with enteric coatings.

Liquid dosage forms for oral administration may include pharmaceutically acceptable emulsions, solutions, suspensions, syrups, and elixirs containing inert diluents commonly used in the art, such as water. Such compositions may also comprise adjuvants, such as wetting agents, emulsifying and suspending agents, cyclodextrins, and sweetening, flavoring, and perfuming agents.

In some embodiments, the formulation is suitable for intravenous administration. In other embodiments, the formulation is suitable for intramuscular administration. Formulations suitable for intravenous and intramuscular administration include aqueous and non-aqueous, isotonic sterile injection solutions, which can contain anti-oxidants, buffers, bacteriostats, and solutes that render the formulation compatible with the blood of the intended recipient, and aqueous and non-aqueous sterile suspensions that can include suspending agents, solubilizers, thickening agents, stabilizers, and preservatives. The formulations can be presented in unit-dose or multi-dose sealed containers, such as ampules and vials, and can be stored in a freeze-dried (lyophilized) condition requiring only the addition of the sterile liquid excipient, for example, water, for injections, immediately prior to use. Extemporaneous injection solutions and suspensions can be prepared from sterile powders, granules, and tablets of the kind previously described.

In some embodiments, the ratio by weight of the cidofovir and the other antiviral agent in the pharmaceutical composition is about 1 to 1. In some embodiments, the weight ratio may be between about 0.001 to about 1 and about 1000 to about 1, or between about 0.01 to about 1 and 100 to about 1. In some embodiments, the ratio by weight of the cidofovir and the other antiviral is less than any of about 1000:1, 900:1, 800:1, 700:1, 600:1, 500:1, 400:1, 300:1, 200:1, 100:1, 50:1, 30:1, 10:1, 9:1, 8:1, 7:1, 6:1, 5:1, 4:1, 3:1, 2:1, and 1:1. In some embodiments, the ratio by weight of the cidofovir and the other antiviral is more than any of about 1:1, 2:1, 3:1, 4:1, 5:1, 6:1, 7:1, 8:1, 9:1, 10:1, 30:1, 50:1, 75:1, 100:1, 200:1, 300:1, 400:1, 500:1, 600:1, 700:1, 800:1, 900:1, and 1000:1. In some embodiments, the ratio by weight of the cidofovir and the other antiviral is less than any of about 1:1, 1:2, 1:3, 1:4, 1:5, 1:6, 1:7, 1:8, 1:9, 1:10, 1:30, 1:50, 1:100, 1:200, 1:300, 1:400, 1:500, 1:600, 1:700, 1:800, 1:900, and 1:1000. In other embodiments, the ratio by weight of the cidofovir and the other antiviral is more than any of about 1:1000, 1:900, 1:800, 1:700, 1:600, 1:500, 1:400, 1:300, 1:200, 1:100, 1:50, 1:30, 1:10, 1:9, 1:8, 1:7, 1:6, 1:5, 1:4, 1:3, 1:2, and 1:1. Other ratios are contemplated.

In some embodiments, there is provided a pharmaceutical composition comprising: a) cidofovir, and b) zidovudine. In some embodiments, the composition is suitable for intravenous administration. In other embodiments, the composition is suitable for oral administration. In other embodiments, the composition is suitable for intramuscular administration. In some embodiments, the weight ratio of the cidofovir to zidovudine in the pharmaceutical composition is about any of 10:1, 9:2, 8:1, 7:1, 6:1, 5:1. 4:1, 3:1, 2:1, 1:1, 1:2, 1:3, 1:4, 1:5, 1:6, 1:7, 1:8, 1:9, or 1:10.

In some embodiments, there is provided a pharmaceutical composition comprising: a) cidofovir, and b) stavudine. In some embodiments, the composition is suitable for intravenous administration. In other embodiments, the composition is suitable for oral administration. In other embodiments, the composition is suitable for intramuscular administration. In some embodiments, the weight ratio of the cidofovir to stavudine in the pharmaceutical composition is about any of 10:1, 9:2, 8:1, 7:1, 6:1, 5:1. 4:1, 3:1, 2:1, 1:1, 1:2, 1:3, 1:4, 1:5, 1:6, 1:7, 1:8, 1:9, or 1:10.

In some embodiments, there is provided a pharmaceutical composition comprising: a) cidofovir, and b) valacyclovir. In some embodiments, the composition is suitable for intravenous administration. In other embodiments, the composition is suitable for oral administration. In other embodiments, the composition is suitable for intramuscular administration. In some embodiments, the weight ratio of the cidofovir to valganciclovir in the pharmaceutical composition is about any of 10:1, 9:2, 8:1, 7:1, 6:1, 5:1. 4:1, 3:1, 2:1, 1:1, 1:2, 1:3, 1:4, 1:5, 1:6, 1:7, 1:8, 1:9, or 1:10.

Also within the scope of the present disclosure is a kit comprising a combination therapy described herein. The kit can include one or more other elements including: instructions for use; devices or other materials for preparing the compositions for administration; pharmaceutically acceptable carriers; and devices or other materials for administration to a subject.

The combination therapies disclosed herein have in vitro and in vivo therapeutic and/or prophylactic utilities. For example, the compositions disclosed herein can be administered to cells in culture, in vitro or ex vivo, or to a subject, e.g., a human subject, to treat, prevent, and/or diagnose a variety of disorders, such as viral infection, e.g., SARS-CoV-2 infection.

Treatment with Nevirapine

Methods

Provided herein are methods for treating and/or preventing coronavirus-associated infection in an individual comprising administering to the individual an effective amount of nevirapine and an effective amount of a second antiviral agent. In some embodiments, the second antiviral agent is a reverse transcriptase inhibitor. In other embodiments the other antiviral agent is a protease inhibitor. In other embodiments, the other antiviral agent is an integrase inhibitor. In other embodiments, the other antiviral agent is a viral DNA polymerase inhibitor. In some embodiments, the second antiviral agent is valacyclovir. In some embodiments, the second antiviral agent is etravirine.

Certain aspects of the present disclosure relate to viral infections. In some embodiments, the virus is an enveloped virus. Examples of enveloped viruses are well known in the art and include, without limitation, the virus families of Arenavirus, Arterivirus, Asfarvirus, Baculovirus, Bunyavirus, Coronavirus, Cystovirus, Deltavirus, Filovirus, Flavivirus, Fusellovirus, Hepadnavirus, Herpesvirus, Iridovirus, Lipothrixivirus, Orthomyxovirus, Paramyxovirus, Plasmavirus, Polydnavirus, Poxvirus, Retrovirus, Rhabdovirus, and Togavirus. In some embodiments, the virus is a Coronavirus, e.g., severe acute respiratory syndrome coronavirus 1 (SARS-CoV-1), severe acute respiratory syndrome coronavirus 2 (SARS-CoV-2), or Middle East respiratory syndrome-related coronavirus (MERS-CoV). In some embodiments, the virus is an Orthomyxovirus, e.g., influenza virus A, B, or C. In some embodiments, the virus is an Orthopneumovirus, e.g., respiratory syncytial virus (RSV).

In some embodiments, an individual has been diagnosed with a coronavirus-associated disease. In some embodiments, an individual has been diagnosed with Severe Acute Respiratory Syndrome Coronavirus 2 (COVID-19). In some embodiments, an individual has or has been diagnosed with acute respiratory distress syndrome (ARDS), e.g., prior to treatment with the combination therapy. Methods for diagnosis of ARDS include, without limitation, chest X-ray, CT scanning, and/or measurement of oxygen levels. In some embodiments, an individual has been diagnosed with an Angiotensin-Converting Enzyme 2 (ACE2)-associated disease. In some embodiments, an individual has been diagnosed with Severe Acute Respiratory Syndrome (SARS). In some embodiments, an individual has been diagnosed with Middle East Respiratory Syndrome (MERS).

In some embodiments, an individual is treated with a combination of nevirapine and etravirine. In some embodiments, the nevirapine is administered orally. In some embodiments, the nevirapine is administered intramuscularly. In some embodiments, the nevirapine is administered orally.

In some embodiments, an individual is treated with a combination of nevirapine and etravirine. In some embodiments, the nevirapine is administered orally. In some embodiments, the nevirapine is administered intramuscularly. In some embodiments, the nevirapine is administered orally.

In some embodiments, the valacyclovir is administered orally. In some embodiments, the valacyclovir is administered intramuscularly. In other embodiments, the valacyclovir is administered intravenously. In some embodiments, the nevirapine and the valacyclovir are administered simultaneously.

In some embodiments, the etravirine is administered orally. In some embodiments, the etravirine is administered intramuscularly. In other embodiments, the etravirine is administered intravenously. In some embodiments, the nevirapine and the etravirine are administered simultaneously.

In some embodiments, the nevirapine and the valacyclovir are administered in a single composition. In some embodiments, the single composition is administered orally. In some embodiments, the single composition is administered intravenously. In some embodiments, the single composition is administered intramuscularly.

In some embodiments, the nevirapine and the etravirine are administered in a single composition. In some embodiments, the single composition is administered orally. In some embodiments, the single composition is administered intravenously. In some embodiments, the single composition is administered intramuscularly.

The composition comprising nevirapine and the valacyclovir can be administered simultaneously (i.e., simultaneous administration and/or sequentially (i.e., sequential administration)).

The composition comprising nevirapine and the etravirine can be administered simultaneously (i.e., simultaneous administration and/or sequentially (i.e., sequential administration)).

In some embodiments, the nevirapine and the valacyclovir are administered simultaneously. The term “simultaneous administration,” as used herein, means that the nevirapine and the valacyclovir agent are administered with a time separation no more than about 15 minutes(s), such as no more than about any of 10, 5, or 1 minutes. When the drugs are administered simultaneously, the nevirapine and valacyclovir may be contained in the same composition (e.g., a composition comprising the nevirapine and the valacyclovir) or in separate compositions (e.g., the nevirapine is contained in one composition and the valacyclovir is contained in another composition).

In some embodiments, the nevirapine and the etravirine are administered simultaneously. When the drugs are administered simultaneously, the nevirapine and etravirine may be contained in the same composition (e.g., a composition comprising the nevirapine and the etravirine) or in separate compositions (e.g., the nevirapine is contained in one composition and the etravirine is contained in another composition).

In some embodiments, the nevirapine and the valacyclovir are administered sequentially. The term “sequential administration” as used herein means that the nevirapine and the valacyclovir are administered with a time separation of more than about 15 minutes, such as more than any of 20, 30, 40, 50, or more minutes. Either the nevirapine or the valacyclovir may be administered first. In some embodiments, the nevirapine is administered prior to administration of the valacyclovir. In some embodiments, the nevirapine is administered following administration of the valacyclovir. The nevirapine and the valacyclovir are contained in separate compositions, which may be contained in the same or different packages.

In some embodiments, the nevirapine and the etravirine are administered sequentially. Either the nevirapine or the etravirine may be administered first. In some embodiments, the nevirapine is administered prior to administration of the etravirine. In some embodiments, the nevirapine is administered following administration of the etravirine. The nevirapine and the etravirine are contained in separate compositions, which may be contained in the same or different packages.

In some embodiments, the individual is a human. In some embodiments, the individual suffers from a pre-existing health condition correlated with poor prognosis following SARS-CoV2-disease. In some embodiments, the pre-existing health condition is cancer. In some embodiments, the pre-existing health condition is chronic kidney disease. In some embodiments, the pre-existing health condition is chronic obstructive pulmonary disease. In some embodiments, the pre-existing health condition is Down Syndrome. In some embodiments, the pre-existing health condition is a heart condition. In some embodiments, the pre-existing health condition is heart failure. In some embodiments, the pre-existing health condition is coronary artery disease. In some embodiments, the pre-existing health condition is cardiomyopathy. In some embodiments, the pre-existing health condition is an immunocompromised state. In some embodiments, the pre-existing health condition is obesity. In some embodiments, the pre-existing health condition is pregnancy. In some embodiments, the pre-existing health condition is sickle cell disease. In some embodiments, the pre-existing health condition is smoking. In some embodiments, the pre-existing health condition is Type I diabetes mellitus. In some embodiments, the pre-existing health condition is Type 2 diabetes mellitus. In some embodiments, the pre-existing health condition is asthma. In some embodiments, the pre-existing health condition is cerebrovascular disease. In some embodiments, the pre-existing health condition is cystic fibrosis. In some embodiments, the pre-existing health condition is hypertension. In some embodiments, the pre-existing health condition is a neurologic condition. In some embodiments, the pre-existing health condition is liver disease. In some embodiments, the pre-existing health condition is pulmonary fibrosis. In some embodiments, the pre-existing health condition is thalassemia. In some embodiments, the pre-existing health condition is 65 years or greater of age.

In some embodiments, the nevirapine and the valacyclovir or the nevirapine and the etravirine is accompanied by a second therapy administered to the individual. In some embodiments, the second therapy is remdesivir. In some embodiments, the second therapy is monoclonal antibody. In some embodiments, the monoclonal antibody is targeted against SARS-CoV-2. In some embodiments, the second therapy is casirivimab and imdevimab. In some embodiments, the second therapy is administered intravenously. In some embodiments, the second therapy is mechanical ventilation.

Nevirapine

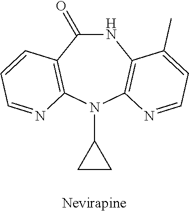 [SMILES: Cc1ccnc2c1NC(=O)c1cccnc1N2C1CC1]

Nevirapine is an antiviral compound that belongs to the class of reverse transcriptase inhibitors. Reverse transcriptase inhibitors are a class of anti-viral drug that is used to treat HIV/AIDS. Reverse transcriptase inhibitors inhibit the activity of reverse transcriptase, an enzyme required for the replication of retroviruses. Nevirapine is a non-nucleoside reverse transcriptase inhibitor. Non-nucleoside reverse transcriptase inhibitors function through binding directly to the reverse transcriptase enzyme, thereby altering its conformation to prevent DNA binding.

Nevirapine is administered orally and has been approved for the treatment of HIV/AIDS. In some embodiments, the nevirapine is administered intravenously. In other embodiments, the nevirapine is administered intramuscularly. In other embodiments, the nevirapine is administered orally. In some embodiments, the individual is administered about 200 mg orally once daily for 14 days. In other embodiments, the individual is administered about 50, 100, 150, 200, 250, 300, 350, 400, 450, 500, 550, 600, 650, 700, 750, 800, 850, 900, 950, or 1000 mg nevirapine. In other embodiments, the individual is administered about 1500-2500 mg or more of nevirapine.

Valacyclovir

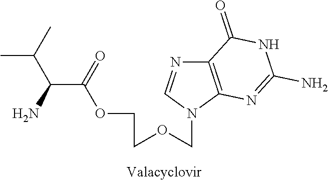 [SMILES: CC(C)[C@H](N)C(=O)OCCOCn1cnc2c(=O)[nH]c(N)nc21]

Valacyclovir is an antiviral compound that belongs to the class of viral DNA polymerase inhibitors. Viral DNA polymerases function through inhibition of the viral DNA polymerase, which prevents viral genome replication and transcription.

Valacyclovir is approved for treating outbreaks of herpes simplex or herpes zoster (shingles), and it can also be used to prevent cytomegalovirus following kidney transplant. In some embodiments, the valacyclovir is administered intravenously. In some embodiments, the valacyclovir is administered intramuscularly. In some embodiments, the valacyclovir is administered orally. In some embodiments, the valacyclovir is administered 1 gram twice daily for 10 days. In some embodiments, the individual is administered about 0.25, 0.5, 0.75, 1, 2, 3, 4, or 5 grams valacyclovir. In other embodiments, the individual is administered 5-8 grams valacyclovir or more.

Etravirine

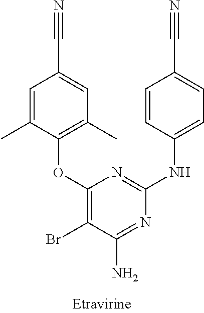 [SMILES: Cc1cc(C#N)cc(C)c1Oc1nc(Nc2ccc(C#N)cc2)nc(N)c1Br]

Etravirine is an antiviral compound that belongs to the class of reverse transcriptase inhibitors. Etravirine inhibits reverse transcriptase by binding at a non-substrate site on the enzyme, different than that for nucleoside analog reverse transcriptase inhibitors (NRTIs) and nucleotide analog reverse transcriptase inhibitors (NtRTIs). Upon binding to the enzyme at a site proximal to the polymerase active site, etravirine can inhibit the movement of protein domains that are needed to carry out the process of DNA synthesis.

Etravirine is currently approved for human immunodeficiency virus (HIV) infection. In some embodiments, the etravirine is administered intravenously. In some embodiments, the etravirine is administered intramuscularly. In some embodiments, the etravirine is administered orally. In some embodiments, the etravirine is administered 0.2 gram twice daily. In some embodiments, the individual is administered about 0.05, 0.1, 0.15, 0.2, 0.25, 0.3, 0.4, 0.5, 1, 2, 3, 4, or 5 grams etravirine. In other embodiments, the individual is administered 1-5 grams of etravirine.

Exemplary Embodiments for Combination Therapy of Cidofovir with Other Antiviral Agent

In some embodiments, there is provided a method of treating an infectious disease in an individual comprising administering to an individual: a) an effective amount of nevirapine, and b) an effective amount of valacyclovir. In some embodiments, the valacyclovir is administered intravenously. In some embodiments, the valacyclovir is administered intramuscularly. In some embodiments, the valacyclovir is administered orally. In some embodiments, the valacyclovir is administered 1 gram twice daily for 10 days. In some embodiments, the individual is administered about 0.25, 0.5, 0.75, 1, 2, 3, 4, or 5 grams valacyclovir. In other embodiments, the individual is administered 5-8 grams valacyclovir or more. In some embodiments, the nevirapine is administered intravenously. In other embodiments, the nevirapine is administered intramuscularly. In other embodiments, the nevirapine is administered orally. In some embodiments, the individual is administered about 200 mg orally once daily for 14 days. In other embodiments, the individual is administered about 50, 100, 150, 200, 250, 300, 350, 400, 450, 500, 550, 600, 650, 700, 750, 800, 850, 900, 950, or 1000 mg nevirapine. In other embodiments, the individual is administered about 1500-2500 mg or more of nevirapine.

In some embodiments, there is a pharmaceutical composition comprising nevirapine and valacyclovir.

In some embodiments, there is provided a method of treating an infectious disease in an individual comprising administering to an individual: a) an effective amount of nevirapine, and b) an effective amount of etravirine. In some embodiments, the etravirine is administered intravenously. In some embodiments, the etravirine is administered intramuscularly. In some embodiments, the etravirine is administered orally. In some embodiments, the etravirine is administered 0.2 gram twice daily. In some embodiments, the individual is administered about 0.05, 0.1, 0.15, 0.2, 0.25, 0.3, 0.4, 0.5, 1, 2, 3, 4, or 5 grams etravirine. In other embodiments, the individual is administered 1-5 grams etravirine or more. In some embodiments, the nevirapine is administered intravenously. In other embodiments, the nevirapine is administered intramuscularly. In other embodiments, the nevirapine is administered orally. In some embodiments, the individual is administered about 200 mg orally once daily for 14 days. In other embodiments, the individual is administered about 50, 100, 150, 200, 250, 300, 350, 400, 450, 500, 550, 600, 650, 700, 750, 800, 850, 900, 950, or 1000 mg nevirapine. In other embodiments, the individual is administered about 1500-2500 mg or more of nevirapine.

In some embodiments, there is a pharmaceutical composition comprising nevirapine and etravirine.

Pharmaceutical Compositions

Also provided are pharmaceutical compositions comprising nevirapine and valacyclovir. Pharmaceutical compositions containing the compounds of the present disclosure may be in any form suitable for the intended method of administration. In some embodiments, the pharmaceutical composition is suitable for oral administration. Formulations suitable for oral administration can consist of (a) liquid solutions, such as an effective amount of the compound dissolved in diluents, such as water, saline, or orange juice, (b) capsules, sachets or tablets, each containing a predetermined amount of the active ingredient, as solids or granules, (c) suspensions in an appropriate liquid, and (d) suitable emulsions. Tablet forms can include one or more of lactose, mannitol, corn starch, potato starch, microcrystalline cellulose, acacia, gelatin, colloidal silicon dioxide, croscarmellose sodium, talc, magnesium stearate, stearic acid, and other excipients, colorants, diluents, buffering agents, moistening agents, preservatives, flavoring agents, and pharmacologically compatible excipients. Lozenge forms can comprise the active ingredient in a flavor, usually sucrose and acacia or tragacanth, as well as pastilles comprising the active ingredient in an inert base, such as gelatin and glycerin, or sucrose and acacia, emulsions, gels, and the like containing, in addition to the active ingredient, such excipients as are known in the art.

Examples of suitable carriers, excipients, and diluents include, but are not limited to, lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, saline solution, syrup, methylcellulose, methyl- and propylhydroxybenzoates, talc, magnesium stearate, and mineral oil. The formulations can additionally include lubricating agents, wetting agents, emulsifying and suspending agents, preserving agents, sweetening agents or flavoring agents.

Solid dosage forms for oral administration may include capsules, tablets, pills, powders and granules. In such solid dosage forms, the active compound may be admixed with at least one inert diluent such as sucrose, lactose, or starch. Such dosage forms may also comprise additional substances other than inert diluents, e.g., lubricating agents such as magnesium stearate. In the case of capsules, tablets, and pills, the dosage forms may also comprise buffering agents. Tablets and pills can additionally be prepared with enteric coatings.

Liquid dosage forms for oral administration may include pharmaceutically acceptable emulsions, solutions, suspensions, syrups, and elixirs containing inert diluents commonly used in the art, such as water. Such compositions may also comprise adjuvants, such as wetting agents, emulsifying and suspending agents, cyclodextrins, and sweetening, flavoring, and perfuming agents.

In some embodiments, the formulation is suitable for intravenous administration. In other embodiments, the formulation is suitable for intramuscular administration. Formulations suitable for intravenous and intramuscular administration include aqueous and non-aqueous, isotonic sterile injection solutions, which can contain anti-oxidants, buffers, bacteriostats, and solutes that render the formulation compatible with the blood of the intended recipient, and aqueous and non-aqueous sterile suspensions that can include suspending agents, solubilizers, thickening agents, stabilizers, and preservatives. The formulations can be presented in unit-dose or multi-dose sealed containers, such as ampules and vials, and can be stored in a freeze-dried (lyophilized) condition requiring only the addition of the sterile liquid excipient, for example, water, for injections, immediately prior to use. Extemporaneous injection solutions and suspensions can be prepared from sterile powders, granules, and tablets of the kind previously described.

In some embodiments, the ratio by weight of the nevirapine and the valacyclovir in the pharmaceutical composition is about 1 to 1. In some embodiments, the weight ratio may be between about 0.001 to about 1 and about 1000 to about 1, or between about 0.01 to about 1 and 100 to about 1. In some embodiments, the ratio by weight of the nevirapine and the valacyclovir is less than any of about 1000:1, 900:1, 800:1, 700:1, 600:1, 500:1, 400:1, 300:1, 200:1, 100:1, 50:1, 30:1, 10:1, 9:1, 8:1, 7:1, 6:1, 5:1, 4:1, 3:1, 2:1, and 1:1. In some embodiments, the ratio by weight of the nevirapine and the valacyclovir is more than any of about 1:1, 2:1, 3:1, 4:1, 5:1, 6:1, 7:1, 8:1, 9:1, 10:1, 30:1, 50:1, 75:1, 100:1, 200:1, 300:1, 400:1, 500:1, 600:1, 700:1, 800:1, 900:1, and 1000:1. In some embodiments, the ratio by weight of the nevirapine and the valacyclovir is less than any of about 1:1, 1:2, 1:3, 1:4, 1:5, 1:6, 1:7, 1:8, 1:9, 1:10, 1:30, 1:50, 1:100, 1:200, 1:300, 1:400, 1:500, 1:600, 1:700, 1:800, 1:900, and 1:1000. In other embodiments, the ratio by weight of the nevirapine and the valacyclovir is more than any of about 1:1000, 1:900, 1:800, 1:700, 1:600, 1:500, 1:400, 1:300, 1:200, 1:100, 1:50, 1:30, 1:10, 1:9, 1:8, 1:7, 1:6, 1:5, 1:4, 1:3, 1:2, and 1:1. Other ratios are contemplated.

In some embodiments, the ratio by weight of the nevirapine and the etravirine in the pharmaceutical composition is about 1 to 1. In some embodiments, the weight ratio may be between about 0.001 to about 1 and about 1000 to about 1, or between about 0.01 to about 1 and 100 to about 1. In some embodiments, the ratio by weight of the nevirapine and the etravirine is less than any of about 1000:1, 900:1, 800:1, 700:1, 600:1, 500:1, 400:1, 300:1, 200:1, 100:1, 50:1, 30:1, 10:1, 9:1, 8:1, 7:1, 6:1, 5:1, 4:1, 3:1, 2:1, and 1:1. In some embodiments, the ratio by weight of the nevirapine and the etravirine is more than any of about 1:1, 2:1, 3:1, 4:1, 5:1, 6:1, 7:1, 8:1, 9:1, 10:1, 30:1, 50:1, 75:1, 100:1, 200:1, 300:1, 400:1, 500:1, 600:1, 700:1, 800:1, 900:1, and 1000:1. In some embodiments, the ratio by weight of the nevirapine and the etravirine is less than any of about 1:1, 1:2, 1:3, 1:4, 1:5, 1:6, 1:7, 1:8, 1:9, 1:10, 1:30, 1:50, 1:100, 1:200, 1:300, 1:400, 1:500, 1:600, 1:700, 1:800, 1:900, and 1:1000. In other embodiments, the ratio by weight of the nevirapine and the etravirine is more than any of about 1:1000, 1:900, 1:800, 1:700, 1:600, 1:500, 1:400, 1:300, 1:200, 1:100, 1:50, 1:30, 1:10, 1:9, 1:8, 1:7, 1:6, 1:5, 1:4, 1:3, 1:2, and 1:1. Other ratios are contemplated.

In some embodiments, there is provided a pharmaceutical composition comprising: a) nevirapine, and b) valacyclovir. In some embodiments, the composition is suitable for intravenous administration. In other embodiments, the composition is suitable for oral administration. In other embodiments, the composition is suitable for intramuscular administration. In some embodiments, the weight ratio of the nevirapine to valganciclovir in the pharmaceutical composition is about any of 10:1, 9:2, 8:1, 7:1, 6:1, 5:1. 4:1, 3:1, 2:1, 1:1, 1:2, 1:3, 1:4, 1:5, 1:6, 1:7, 1:8, 1:9, or 1:10.

Also within the scope of the present disclosure is a kit comprising a combination therapy described herein. The kit can include one or more other elements including: instructions for use; devices or other materials for preparing the compositions for administration; pharmaceutically acceptable carriers; and devices or other materials for administration to a subject.

The combination therapies disclosed herein have in vitro and in vivo therapeutic and/or prophylactic utilities. For example, the compositions disclosed herein can be administered to cells in culture, in vitro or ex vivo, or to a subject, e.g., a human subject, to treat, prevent, and/or diagnose a variety of disorders, such as viral infection, e.g., SARS-CoV-2 infection.

### Patient Population

In some embodiments, the individual is a human. In some embodiments, the individual is a male. In some embodiments, the individual is a female. In some embodiments, the individual is at least about any of 12, 24, 36, 48, 50, 55, 60, 65, 70, 75, or 80 years old, or older. In some embodiments, the individual is no more than about 60, 50, 40, 30, 20, or 10 years old, or younger. In some embodiments, the individual is clinically obese. In some embodiments, the individual is overweight. In other embodiments, the individual is normal weight. In other embodiments, the individual is underweight. In some embodiments, the individual has a body mass index (BMI) of about >30. In other embodiments, the individual has a BMI between about 25 and 30. In other embodiments, the individual has a BMI between bout 18.5 and 25. In other embodiments, the individual has a BMI under about 18.5.

In some embodiments, the individual suffers from a pre-existing health condition correlated with poor prognosis following SARS-CoV2-disease. In some embodiments, the pre-existing health condition is cancer. In some embodiments, the pre-existing health condition is chronic kidney disease. In some embodiments, the pre-existing health condition is chronic obstructive pulmonary disease. In some embodiments, the pre-existing health condition is Down Syndrome. In some embodiments, the pre-existing health condition is a heart condition. In some embodiments, the pre-existing health condition is heart failure. In some embodiments, the pre-existing health condition is coronary artery disease. In some embodiments, the pre-existing health condition is cardiomyopathy. In some embodiments, the pre-existing health condition is an immunocompromised state. In some embodiments, the pre-existing health condition is obesity. In some embodiments, the pre-existing health condition is pregnancy. In some embodiments, the pre-existing health condition is sickle cell disease. In some embodiments, the pre-existing health condition is smoking. In some embodiments, the pre-existing health condition is Type I diabetes mellitus. In some embodiments, the pre-existing health condition is Type 2 diabetes mellitus. In some embodiments, the pre-existing health condition is asthma. In some embodiments, the pre-existing health condition is cerebrovascular disease. In some embodiments, the pre-existing health condition is cystic fibrosis. In some embodiments, the pre-existing health condition is hypertension. In some embodiments, the pre-existing health condition is a neurologic condition. In some embodiments, the pre-existing health condition is liver disease. In some embodiments, the pre-existing health condition is pulmonary fibrosis. In some embodiments, the pre-existing health condition is thalassemia. In some embodiments, the pre-existing health condition is 65 years or greater of age.

### EXEMPLARY EMBODIMENTS

Embodiment 1a. A method of treating an infectious disease in an individual comprising administering to an individual:
a) an effective amount of peramivir, and
b) an effective amount of at least one other antiviral agent, wherein the other antiviral agent is selected from the group consisting of a reverse transcriptase inhibitor, a protease inhibitor, an integrase inhibitor, and a viral DNA polymerase inhibitor.

Embodiment 2a. The method of Embodiment 1a, wherein the infectious disease is a coronavirus-associated disease.

Embodiment 3a. The method of Embodiment 2a, wherein the coronavirus-associated disease is Severe Acute Respiratory Syndrome Coronavirus 2 (COVID-19), an Angiotensin-Converting Enzyme 2 (ACE2)-associated disease, Acute Respiratory Distress Syndrome (ARDS), Severe Acute Respiratory Syndrome (SARS), or Middle East Respiratory Syndrome (MERS).

Embodiment 4a. The method according to any one of Embodiments 1a-3a, wherein the other antiviral agent is a reverse transcriptase inhibitor.

Embodiment 5a. The method according to Embodiment 4a, wherein the reverse transcriptase inhibitor is a nucleoside analog reverse transcriptase inhibitor.

Embodiment 6a. The method according to Embodiment 5a, wherein the nucleoside analog reverse transcriptase inhibitor is abacavir, didanosine, or stavudine.

Embodiment 7a. The method according to any one of Embodiments 1a-3a, wherein the reverse transcriptase inhibitor is a non-nucleoside reverse transcriptase inhibitor.

Embodiment 8a. The method according to Embodiment 7a, wherein the non-nucleoside reverse transcriptase inhibitor is etravirine or rilpivirine.

Embodiment 9a. The method according to any one of Embodiments 1a-3a, wherein the other antiviral agent is a protease inhibitor.

Embodiment 10a. The method according to Embodiment 9a, wherein the protease inhibitor is darunavir.

Embodiment 11a. The method according to any one of Embodiments 1a-3a, wherein the other antiviral agent is an integrase inhibitor.

Embodiment 12a. The method according to Embodiment 11a, wherein the integrase inhibitor is elvitegravir.

Embodiment 13a. The method according to any one of Embodiments 1a-3a, wherein the other antiviral agent is a viral DNA polymerase inhibitor.

Embodiment 14a. The method according to Embodiment 13a, wherein the viral DNA polymerase inhibitor is acyclovir, ganciclovir, or valganciclovir.

Embodiment 15a. The method according to any one of Embodiments 1a-14a, wherein the peramivir is administered orally.

Embodiment 16a. The method according to any one of Embodiments 1a-14a, wherein the peramivir is administered intramuscularly.

Embodiment 17a. The method according to any one of Embodiments 1a-14a, wherein the peramivir is administered intravenously.

Embodiment 18a. The method according to any one of Embodiments 1a-14a, wherein the other antiviral agent is administered orally.

Embodiment 19a. The method according to any one of Embodiments 1a-14a, wherein the other antiviral agent is administered intramuscularly.

Embodiment 20a. The method according to any one of Embodiments 1a-14a, wherein the other antiviral agent is administered intravenously.

Embodiment 21a. The method according to any one of Embodiments 1a-20a, wherein the peramivir is administered in a one-time dose.

Embodiment 22a. The method according to of Embodiment 6a, wherein the abacavir is administered orally twice daily.

Embodiment 23a. The method according to Embodiment 6a, wherein the abacavir is administered orally once daily.

Embodiment 24a. The method according to Embodiment 6a, wherein the didanosine is administered orally twice daily.

Embodiment 25a. The method according to Embodiment 6a, wherein the stavudine is administered orally every 12 hours.

Embodiment 26a. The method according to Embodiment 8a, wherein the etravirine is administered orally once daily.

Embodiment 27a. The method according to Embodiment 8a, wherein the etravirine is administered orally twice daily.

Embodiment 28a. The method according to Embodiment 8a, wherein the rilpivirine is administered orally once daily.

Embodiment 29a. The method according to Embodiment 8a, wherein the rilpivirine is administered intramuscularly monthly.

Embodiment 30a. The method according to Embodiment 10a, wherein the darunavir is administered orally twice daily.

Embodiment 31a. The method according to Embodiment 12a, wherein the elvitegravir is administered orally once daily.

Embodiment 32a. The method according to Embodiment 14a, wherein the acyclovir is administered intravenously every 8 hours for 5-10 days.

Embodiment 33a. The method according to Embodiment 14a, wherein the ganciclovir is administered intravenously every 12 hours for 14-21 days.

Embodiment 34a. The method according to Embodiment 14a, wherein the ganciclovir is administered orally three times per day.

Embodiment 35a. The method according to Embodiment 14a, wherein the ganciclovir is administered orally six times per day.

Embodiment 36a. The method according to Embodiment 14a, wherein the valganciclovir is administered orally twice daily for 21 days.

Embodiment 37a. The method according to Embodiment 14a, wherein the acyclovir is administered orally five times a day for 5-10 days.

Embodiment 38a. The method according to any one of Embodiments 1a-37a, wherein the peramivir and the other antiviral agent are administered simultaneously.

Embodiment 39a. The method according to Embodiment 38a, wherein the peramivir and the other antiviral agent are administered in a single composition.

Embodiment 40a. The method according to any one of Embodiments 1a-37a, wherein the peramivir and the other antiviral agent are administered sequentially.

Embodiment 41a. The method according to Embodiment 40a, wherein the peramivir is administered prior to administration of the other antiviral agent.

Embodiment 42a. The method according to Embodiment 40a, wherein the peramivir is administered following administration of the other antiviral agent.

Embodiment 43a. The method according to any one of Embodiments 1a-42a, wherein the individual is a human.

Embodiment 44a. The method according to any one of Embodiments 1a-42a, wherein the individual suffers from a pre-existing health condition correlated with poor prognosis following SARS-CoV-2 disease.

Embodiment 45a. The method according to Embodiment 44a, wherein the pre-existing health condition is selected from the group consisting of cancer, chronic kidney disease, chronic obstructive pulmonary disease, Down Syndrome, heart conditions, heart failure, coronary artery disease, cardiomyopathy, immunocompromised states, obesity, pregnancy, sickle cell disease, smoking, Type I diabetes mellitus. Type 2 diabetes mellitus, asthma, cerebrovascular disease, cystic fibrosis, hypertension, neurologic conditions, liver disease, pulmonary fibrosis, thalassemia, and 65 years or greater of age.

Embodiment 46a. The method according to any one of Embodiments 1a-45a, further comprising administering a second therapy to the individual.

Embodiment 47a. The method according to Embodiment 46a, wherein the second therapy comprises remdesivir, monoclonal antibodies, mechanical ventilation, or combinations thereof.

Embodiment 48a. The method according to Embodiment 46a, wherein the second therapy comprises administration of an effective amount of remdesivir.

Embodiment 49a. The method according to Embodiment 46a, wherein the second therapy comprises administration of an effective amount of a monoclonal antibody targeting against SARS-CoV-2.

Embodiment 50a. The method according to Embodiment 49a, wherein the second therapy comprises administration of casirivimab and imdevimab intravenously.

Embodiment 51a. The method according to Embodiment 46a, wherein the second therapy is mechanical ventilation.

Embodiment 52a. A pharmaceutical composition comprising: a) peramivir; and b) at least one other antiviral agent, wherein the other antiviral agent is selected from the group consisting of a reverse transcriptase inhibitor, a protease inhibitor, an integrase inhibitor, and a viral DNA polymerase inhibitor.

Embodiment 53a. The pharmaceutical composition according to Embodiment 52a, wherein the other antiviral agent is a reverse transcriptase inhibitor.

Embodiment 54a. The pharmaceutical composition according to Embodiment 53a, wherein the other reverse transcriptase inhibitor is a nucleoside analog reverse transcriptase inhibitor.

Embodiment 55a. The pharmaceutical composition according to Embodiment 54a, wherein the nucleoside analog reverse transcriptase inhibitor is abacavir, didanosine, or stavudine.

Embodiment 56a. The pharmaceutical composition according to Embodiment 52a, wherein the other reverse transcriptase inhibitor is a non-nucleoside reverse transcriptase inhibitor.

Embodiment 57a. The pharmaceutical composition according to Embodiment 56a, wherein the other non-nucleoside reverse transcriptase inhibitor is etravirine or rilpivirine.

Embodiment 58a. The pharmaceutical composition according to Embodiment 52a, wherein the other antiviral agent is a protease inhibitor.

Embodiment 59a. The pharmaceutical composition according to Embodiment 58a, wherein the other protease inhibitor is darunavir.

Embodiment 60a. The pharmaceutical composition according to Embodiment 52a, wherein the other antiviral agent is an integrase inhibitor.

Embodiment 61a. The pharmaceutical composition according to Embodiment 60a, wherein the other integrase inhibitor is elvitegravir.

Embodiment 62a. The pharmaceutical composition according to Embodiment 52a, wherein the other antiviral agent is a viral DNA polymerase inhibitor.

Embodiment 63a. The pharmaceutical composition according to Embodiment 62a, wherein the other viral DNA polymerase inhibitor is acyclovir, ganciclovir, or valganciclovir.

Embodiment 64a. The pharmaceutical composition of any one of Embodiments 52a-63a, wherein the pharmaceutical composition is a tablet, capsule, or caplet.

Embodiment 65a. The pharmaceutical composition of any one of Embodiments 52a-64a, wherein the pharmaceutical composition is in a vial.

Embodiment 66a. The pharmaceutical composition of any one of Embodiments 52a-63a, wherein the weight ratio of the peramivir and the other antiviral in the composition is about 25:1 to about 1:5.

Embodiment 67a. The pharmaceutical composition of any one of Embodiments 52a-66a, wherein the composition contains about 100-1000 mg peramivir.

Embodiment 68a. The pharmaceutical composition of Embodiment 54a, wherein the nucleoside analog reverse transcriptase inhibitor is abacavir, and wherein the composition contains about 50-1000 mg abacavir.

Embodiment 69a. The pharmaceutical composition of Embodiment 54a, wherein the nucleoside analog reverse transcriptase inhibitor is didanosine, and wherein the composition contains about 50-1000 mg didanosine.

Embodiment 70a. The pharmaceutical composition of Embodiment 54a, wherein the nucleoside analog reverse transcriptase inhibitor is stavudine, and wherein the composition contains about 1-1000 mg stavudine.

Embodiment 71a. The pharmaceutical composition of Embodiment 56a, wherein the non-nucleoside reverse transcriptase inhibitor is etravirine, and wherein the composition contains about 25-1000 mg etravirine.

Embodiment 72a. The pharmaceutical composition of Embodiment 56a, wherein the non-nucleoside reverse transcriptase inhibitor is rilpivirine, and wherein the composition contains about 5-500 ng rilpivirine.

Embodiment 73a. The pharmaceutical composition of Embodiment 56a, wherein the non-nucleoside reverse transcriptase inhibitor is rilpivirine, and wherein the composition contains about 100-1000 mg/mL rilpivirine.

Embodiment 74a. The pharmaceutical composition of Embodiment 59a, wherein the composition contains about 100-1000 ng darunavir.

Embodiment 75a. The pharmaceutical composition of Embodiment 61a, wherein the composition contains about 25-500 mg elvitegravir.

Embodiment 76a. The pharmaceutical composition of Embodiment 62a, wherein the viral DNA polymerase inhibitor is acyclovir, and wherein the composition contains about 25-2000 mg acyclovir.

Embodiment 77a. The pharmaceutical composition of Embodiment 62a, wherein the viral DNA polymerase inhibitor is ganciclovir, and wherein the composition contains about 50-2500 mg ganciclovir.

Embodiment 78a. The pharmaceutical composition of Embodiment 62a, wherein the viral DNA polymerase inhibitor is valganciclovir, and wherein the composition contains about 250-2000 mg of valganciclovir.

Embodiment 79a. The pharmaceutical composition of any one of Embodiments 52a-78a for use in the manufacture of a medicament for treating or preventing an infectious disease in a subject in thereof.

Embodiment 80a. The pharmaceutical composition of any one of Embodiments 52a-79a for use in treating or preventing an infectious disease in a subject in thereof.

Embodiment 81a. Use of the pharmaceutical composition of any one of Embodiments 52a-80a for treating or preventing an infectious disease in a subject in thereof.

Embodiment 82a. A kit, comprising: a) peramivir; b) at least one other antiviral agent, wherein the other antiviral agent is selected from the group consisting of a reverse transcriptase inhibitor, a protease inhibitor, an integrase inhibitor, and a viral DNA polymerase inhibitor; and optionally c) instructions for using a) and b) in combination for treating or preventing an infectious disease in a subject in thereof.

Embodiment 83a. The kit of Embodiment 82a, wherein a) and b) are in separate compositions.

Embodiment 84a. The pharmaceutical composition of Embodiment 79a or 80a, the use in Embodiment 81a, or the kit of Embodiment 82a or 83a, wherein the infectious disease is a coronavirus-associated disease.

Embodiment 85a. The pharmaceutical composition, use, or kit of Embodiment 84a, wherein the coronavirus-associated disease is Severe Acute Respiratory Syndrome Coronavirus 2 (COVID-19), an Angiotensin-Converting Enzyme 2 (ACE2)-associated disease, Acute Respiratory Distress Syndrome (ARDS), Severe Acute Respiratory Syndrome (SARS), or Middle East Respiratory Syndrome (MERS).

Embodiment 1b. A method of treating an infectious disease in an individual comprising administering to an individual: a) an effective amount of cidofovir, and b) an effective amount of at least one other antiviral agent, wherein the other antiviral agent is selected from the group consisting of a reverse transcriptase inhibitor and a viral DNA polymerase inhibitor.

Embodiment 2b. The method of Embodiment 1b, wherein the infectious disease is a coronavirus-associated disease.

Embodiment 3b. The method of Embodiment 2b, wherein the coronavirus-associated disease is Severe Acute Respiratory Syndrome Coronavirus 2 (COVID-19), an Angiotensin-Converting Enzyme 2 (ACE2)-associated disease, Acute Respiratory Distress Syndrome (ARDS), Severe Acute Respiratory Syndrome (SARS), or Middle East Respiratory Syndrome (MERS).

Embodiment 4b. The method according to any one of Embodiments 1b-3b, wherein the other antiviral agent is a reverse transcriptase inhibitor.

Embodiment 5b. The method according to Embodiment 4b, wherein the reverse transcriptase inhibitor is a nucleoside analog reverse transcriptase inhibitor.

Embodiment 6b. The method according to Embodiment 5b, wherein the nucleoside analog reverse transcriptase inhibitor is zidovudine or stavudine.

Embodiment 7b. The method according to any one of Embodiments 1b-3b, wherein the other antiviral agent is a viral DNA polymerase inhibitor.

Embodiment 8b. The method according to Embodiment 7b, wherein the viral DNA polymerase inhibitor is valacyclovir.

Embodiment 9b. The method according to any one of Embodiments 1b-8b, wherein the cidofovir is administered orally.

Embodiment 10b. The method according to any one of Embodiments 1b-8b, wherein the cidofovir is administered intramuscularly.

Embodiment 11b. The method according to any one of Embodiments 1b-8b, wherein the cidofovir is administered intravenously.

Embodiment 12. The method according to any one of Embodiments 1b-8b, wherein the other antiviral agent is administered orally.

Embodiment 13b. The method according to any one of Embodiments 1b-8b, wherein the other antiviral agent is administered intramuscularly.

Embodiment 14b. The method according to any one of Embodiments 1b-8b, wherein the other antiviral agent is administered intravenously.

Embodiment 15b. The method according to any one of Embodiments 1b-14b, wherein the cidofovir is administered in a one-time dose.

Embodiment 16b. The method according to Embodiment 5b, wherein the nucleoside analogue reverse transcriptase inhibitor is zidovudine, and wherein the zidovudine is administered orally once daily.

Embodiment 17b. The method according to Embodiment 5b, wherein the nucleoside analogue reverse transcriptase inhibitor is stavudine, and wherein the stavudine is administered orally every 12 hours.

Embodiment 18b. The method according to Embodiment 8b, wherein the valacyclovir is administered orally twice daily for 10 days.

Embodiment 19b. The method according to any one of Embodiments 1-18b, wherein the cidofovir and the other antiviral agent are administered simultaneously.

Embodiment 20b. The method according to Embodiment 19b, wherein the cidofovir and the other antiviral agent are administered in a single composition.

Embodiment 21b. The method according to any one of Embodiments 1b-18b, wherein the cidofovir and the other antiviral agent are administered sequentially.

Embodiment 22b. The method according to Embodiment 21b, wherein the cidofovir is administered prior to administration of the other antiviral agent.

Embodiment 23b. The method according to Embodiment 21b, wherein the cidofovir is administered following administration of the other antiviral agent.

Embodiment 24b. The method according to any one of Embodiments 1b-23b, wherein the individual is a human.

Embodiment 25b. The method according to any one of Embodiments 1b-23b, wherein the individual suffers from a pre-existing health condition correlated with poor prognosis following S ARS-CoV-2 disease.

Embodiment 26b. The method according to Embodiment 25b, wherein the pre-existing health condition is selected from the group consisting of cancer, chronic kidney disease, chronic obstructive pulmonary disease, Down Syndrome, heart conditions, heart failure, coronary artery disease, cardiomyopathy, immunocompromised states, obesity, pregnancy, sickle cell disease, smoking, Type I diabetes mellitus, Type 2 diabetes mellitus, asthma, cerebrovascular disease, cystic fibrosis, hypertension, neurologic conditions, liver disease, pulmonary fibrosis, thalassemia, and 65 years or greater of age.

Embodiment 27b. The method according to any one of Embodiments 1b-26b, further comprising administering a second therapy to the individual.

Embodiment 28b. The method according to Embodiment 27b wherein the second therapy comprises remdesivir, monoclonal antibodies, mechanical ventilation, or combinations thereof.

Embodiment 29b. The method according to Embodiment 27b, wherein the second therapy comprises administration of an effective amount of remdesivir.

Embodiment 30b. The method according to Embodiment 27b, wherein the second therapy comprises administration of an effective amount of a monoclonal antibody targeting against SARS-CoV-2.

Embodiment 31b. The method according to Embodiment 30b, wherein the second therapy comprises administration of casirivimab and imdevimab intravenously.

Embodiment 32b. The method according to Embodiment 27b, wherein the second therapy is mechanical ventilation.

Embodiment 33b. A pharmaceutical composition comprising: a) cidofovir; and b) at least one other antiviral agent, wherein the other antiviral agent is selected from the group consisting of a reverse transcriptase inhibitor and a viral DNA polymerase inhibitor.

Embodiment 34b. The pharmaceutical composition according to Embodiment 33b, wherein the other antiviral agent is a reverse transcriptase inhibitor.

Embodiment 35b. The pharmaceutical composition according to Embodiment 34b, wherein the other reverse transcriptase inhibitor is a nucleoside analog reverse transcriptase inhibitor.

Embodiment 36b. The pharmaceutical composition according to Embodiment 35b, wherein the other nucleoside analog reverse transcriptase inhibitor is zidovudine or stavudine.

Embodiment 37b. The pharmaceutical composition according to Embodiment 33b, wherein the other antiviral agent is a viral DNA polymerase inhibitor.

Embodiment 38b. The pharmaceutical composition according to Embodiment 37b, wherein the viral DNA polymerase inhibitor is valacyclovir.

Embodiment 39b. The pharmaceutical composition of any one of Embodiments 33b-38b, wherein the pharmaceutical composition is a tablet, capsule, or caplet.

Embodiment 40b. The pharmaceutical composition of any one of Embodiments 33b-39b wherein the pharmaceutical composition is in a vial.

Embodiment 41b. The pharmaceutical composition of any one of Embodiments 33b-38b, wherein the weight ratio of the cidofovir and the other antiviral in the composition is about 25:1 to about 1:5.

Embodiment 42b. The pharmaceutical composition of any one of Embodiments 33b-41b, wherein the composition contains about 25-1000 mg cidofovir.

Embodiment 43b. The pharmaceutical composition of Embodiment 35b, wherein nucleoside analogue reverse transcriptase inhibitor is zidovudine, and wherein the composition contains about 100-1000 mg zidovudine.

Embodiment 44b. The pharmaceutical composition of Embodiment 35b, wherein the nucleoside analog reverse transcriptase inhibitor is stavudine, and wherein the composition contains about 1-1000 mg stavudine.

Embodiment 45b. The pharmaceutical composition of Embodiment 38b, wherein the composition contains about 0.25 to 5 grams valacyclovir.

Embodiment 46b. The pharmaceutical composition of any one of Embodiments 33b-45b for use in the manufacture of a medicament for treating or preventing an infectious disease in a subject in thereof.

Embodiment 47b. The pharmaceutical composition of any one of Embodiments 33b-46b for use in treating or preventing an infectious disease in a subject in thereof.

Embodiment 48b. Use of the pharmaceutical composition of any one of Embodiments 33b-47b for treating or preventing an infectious disease in a subject in thereof.

Embodiment 49b. A kit, comprising: a) cidofovir; b) at least one other antiviral agent, wherein the other antiviral agent is selected from the group consisting of a reverse transcriptase inhibitor, a protease inhibitor, an integrase inhibitor, and a viral DNA polymerase inhibitor; and
optionally c) instructions for using a) and b) in combination for treating or preventing an infectious disease in a subject in thereof.

Embodiment 50b. The kit of Embodiment 49b, wherein a) and b) are in separate compositions.

Embodiment 51b. The pharmaceutical composition of Embodiment 46b or 47b, the use in Embodiment 48b, or the kit of Embodiment 49b or 50b, wherein the infectious disease is a coronavirus-associated disease.

Embodiment 52b. The pharmaceutical composition, use, or kit of Embodiment 51b, wherein the coronavirus-associated disease is Severe Acute Respiratory Syndrome Coronavirus 2 (COVID-19), an Angiotensin-Converting Enzyme 2 (ACE2)-associated disease, Acute Respiratory Distress Syndrome (ARDS), Severe Acute Respiratory Syndrome (SARS), or Middle East Respiratory Syndrome (MERS).

Embodiment 1c. A method of treating an infectious disease in an individual comprising administering to an individual: a) an effective amount of nevirapine, and b) an effective amount of a second antiviral agent, wherein the second antiviral agent is selected from the group consisting of a reverse transcriptase inhibitor, a protease inhibitor, an integrase inhibitor, and a viral DNA polymerase inhibitor.

Embodiment 2c. The method of Embodiment 1c, wherein the second antiviral agent is valacyclovir.

Embodiment 3c. The method of Embodiment 1c, wherein the second antiviral agent is etravirine.

Embodiment 4c. The method of any one of Embodiments 10-3c, wherein the infectious disease is a coronavirus-associated disease.

Embodiment 5c. The method of Embodiment 4c, wherein the coronavirus-associated disease is Severe Acute Respiratory Syndrome Coronavirus 2 (COVID-19), an Angiotensin-Converting Enzyme 2 (ACE2)-associated disease, Acute Respiratory Distress Syndrome (ARDS), Severe Acute Respiratory Syndrome (SARS), or Middle East Respiratory Syndrome (MERS).

Embodiment 6c. The method according to any one of Embodiments 1c-5c, wherein the nevirapine is administered orally.

Embodiment 7c. The method according to any one of Embodiments 1c-5c, wherein the nevirapine is administered intramuscularly.

Embodiment 8c. The method according to any one of Embodiments 1c-5c, wherein the nevirapine is administered intravenously.

Embodiment 9c. The method according to any one of Embodiments 1c-5c, wherein the valacyclovir or the etravirine is administered orally.

Embodiment 10c. The method according to any one of Embodiments 1c-5c, wherein the valacyclovir or the etravirine is administered intramuscularly.

Embodiment 11c. The method according to any one of Embodiments 1c-5c, wherein the valacyclovir or the etravirine is administered intravenously.

Embodiment 12c. The method according to any one of Embodiments 1c-11c, wherein the nevirapine is administered orally once daily for 14 days.

Embodiment 13c. The method according to any one of Embodiments 1c-12c, wherein the valacyclovir is administered orally twice daily for 10 days.

Embodiment 14c. The method according to any one of Embodiments 1c-12c, wherein the etravirine is administered orally twice daily for 14 days.

Embodiment 15c. The method according to any one of Embodiments 1c-13c, wherein the nevirapine and the valacyclovir are administered simultaneously.

Embodiment 16c. The method according to Embodiment 15c, wherein the nevirapine and the valacyclovir are administered in a single composition.

Embodiment 17c. The method according to any one of Embodiments 1c-13c, wherein the nevirapine and the valacyclovir are administered sequentially.

Embodiment 18c. The method according to Embodiment 17c, wherein the nevirapine is administered prior to administration of the valacyclovir.

Embodiment 19c. The method according to Embodiment 17c, wherein the nevirapine is administered following administration of the valacyclovir.

Embodiment 20c. The method according to Embodiment 1c, wherein the nevirapine and the second antiviral agent are administered simultaneously or sequentially.

Embodiment 21c. The method according to any one of Embodiments 1c-12c and 14c, wherein the nevirapine and the etravirine are administered simultaneously.

Embodiment 22c. The method according to Embodiment 21c, wherein the nevirapine and the etravirine are administered in a single composition.

Embodiment 23c. The method according to any one of Embodiments 1c-12c and 14c, wherein the nevirapine and the etravirine are administered sequentially.

Embodiment 24c. The method according to Embodiment 23c, wherein the nevirapine is administered prior to administration of the etravirine.

Embodiment 25c. The method according to Embodiment 23c, wherein the nevirapine is administered following administration of the etravirine.

Embodiment 26c. The method according to any one of Embodiments 1c-25c, wherein the individual is a human.

Embodiment 27c. The method according to any one of Embodiments 1c-25c, wherein the individual suffers from a pre-existing health condition correlated with poor prognosis following SARS-CoV-2 disease.

Embodiment 28c. The method according to Embodiment 18c, wherein the pre-existing health condition is selected from the group consisting of cancer, chronic kidney disease, chronic obstructive pulmonary disease, Down Syndrome, heart conditions, heart failure, coronary artery disease, cardiomyopathy, immunocompromised states, obesity, pregnancy, sickle cell disease, smoking, Type I diabetes mellitus, Type 2 diabetes mellitus, asthma, cerebrovascular disease, cystic fibrosis, hypertension, neurologic conditions, liver disease, pulmonary fibrosis, thalassemia, and 65 years or greater of age.

Embodiment 29c. The method according to any one of Embodiments 1c-28c, further comprising administering a second therapy to the individual.

Embodiment 30c. The method according to Embodiment 29c wherein the second therapy comprises remdesivir, monoclonal antibodies, mechanical ventilation, or combinations thereof.

Embodiment 31c. The method according to Embodiment 30c, wherein the second therapy comprises administration of an effective amount of remdesivir.

Embodiment 32c. The method according to Embodiment 30c, wherein the second therapy comprises administration of an effective amount of a monoclonal antibody targeting against SARS-CoV-2.

Embodiment 33c. The method according to Embodiment 30c, wherein the second therapy comprises administration of casirivimab and imdevimab intravenously.

Embodiment 34c. The method according to Embodiment 30c, wherein the second therapy is mechanical ventilation.

Embodiment 35c. A pharmaceutical composition comprising: a) nevirapine; and b) a reverse transcriptase inhibitor, a protease inhibitor, an integrase inhibitor, and a viral DNA polymerase inhibitor.

Embodiment 36c. A pharmaceutical composition comprising: a) nevirapine; and b) valacyclovir and/or etravirine.

Embodiment 37c. The pharmaceutical composition of Embodiment 35c or 36c, wherein the pharmaceutical composition is a tablet, capsule, or caplet.

Embodiment 38c. The pharmaceutical composition of Embodiment 35c or 36c wherein the pharmaceutical composition is in a vial.

Embodiment 39c. The pharmaceutical composition of Embodiment 35c or 36c, wherein the weight ratio of the nevirapine and the valacyclovir/etravirine in the composition is about 25:1 to about 1:25.

Embodiment 40c. The pharmaceutical composition of any one of Embodiments 35c-39c, wherein the composition contains about 50-1000 mg nevirapine.

Embodiment 41c. The pharmaceutical composition any one of Embodiments 35c-40c, wherein the composition contains about 0.25 to 5 grams valacyclovir.

Embodiment 42c. The pharmaceutical composition any one of Embodiments 35c-41c, wherein the composition contains about 0.1 to 5 grams etravirine.

Embodiment 43c. The pharmaceutical composition of any one of Embodiments 35c-42c for use in the manufacture of a medicament for treating or preventing an infectious disease in a subject in thereof.

Embodiment 44c. The pharmaceutical composition of any one of Embodiments 35c-43c for use in treating or preventing an infectious disease in a subject in thereof.

Embodiment 45c. Use of the pharmaceutical composition of any one of Embodiments 35c-44c for treating or preventing an infectious disease in a subject in thereof.

Embodiment 46c. A kit, comprising: a) nevirapine; b) at least one other antiviral agent, wherein the other antiviral agent is selected from the group consisting of a reverse transcriptase inhibitor, a protease inhibitor, an integrase inhibitor, and a viral DNA polymerase inhibitor; and
optionally c) instructions for using a) and b) in combination for treating or preventing an infectious disease in a subject in thereof.

Embodiment 47c. The kit of Embodiment 46c, wherein a) and b) are in separate compositions.

Embodiment 48c. The kit of Embodiment 46c or 47c, wherein the at least one other antiviral agent is valacyclovir and/or etravirine.

Embodiment 49c. The pharmaceutical composition of Embodiment 43c or 44c, the use in Embodiment 45c, or the kit of any one of Embodiments 46c-48c, wherein the infectious disease is a coronavirus-associated disease.

Embodiment 50c. The pharmaceutical composition, use, or kit of Embodiment 49c, wherein the coronavirus-associated disease is Severe Acute Respiratory Syndrome Coronavirus 2 (COVID-19), an Angiotensin-Converting Enzyme 2 (ACE2)-associated disease, Acute Respiratory Distress Syndrome (ARDS), Severe Acute Respiratory Syndrome (SARS), or Middle East Respiratory Syndrome (MERS).

### EXAMPLES

The following example is provided to illustrate, but not limit, the present disclosure.

### Example 1

### In Vitro Screening of Combined Antiviral Activity: Screen Studies in an In Vitro Model of Inhibiting SARS-CoV-2 Growth and Proliferation

Combinations of antiviral agents were shown to inhibit the growth of SARS-CoV-2 in vitro. Two 96-well plates were seeded with Vero (African green monkey kidney epithelial) cells. The cells were then infected with SARS-CoV-2 virus and treated with 192 different antiviral agent combinations. The antiviral agent combinations were introduced to the 96-well plate cell cultures using pin-array technology.

The individual wells of each plate were then analyzed using florescence microscopy to measure inhibition of growth and proliferation of the SARS-CoV-2 virus in vitro. Both the nucleus of the Vero cells and the SARS-CoV-2 nucleocapsid protein were imaged. The average intensity of the nucleocapsid protein per cell was measured and quantified for each antiviral agent combination.

The disclosures of all publications, patents, patent applications and published patent applications referred to herein by an identifying citation are hereby incorporated herein by reference in their entirety.

The present disclosure is not intended to be limited in scope to the particular disclosed embodiments, which are provided, for example, to illustrate various aspects of the present disclosure. Various modifications to the compositions and methods described will become apparent from the description and teachings herein. Such variations may be practiced without departing from the true scope and spirit of the disclosure and are intended to fall within the scope of the present disclosure.

1. A method of treating an infectious disease in an individual comprising administering to an individual:
a) an effective amount of peramivir, cidofovir, or nevirapine, and
b) an effective amount of at least one other antiviral agent, wherein the other antiviral agent is selected from the group consisting of a reverse transcriptase inhibitor, a protease inhibitor, an integrase inhibitor, and a viral DNA polymerase inhibitor.

2. The method of claim 1, wherein the infectious disease is a coronavirus-associated disease.

3. The method of claim 2, wherein the coronavirus-associated disease is Severe Acute Respiratory Syndrome Coronavirus 2 (COVID-19), an Angiotensin-Converting Enzyme 2 (ACE2)-associated disease, Acute Respiratory Distress Syndrome (ARDS), Severe Acute Respiratory Syndrome (SARS), or Middle East Respiratory Syndrome (MERS).

4. The method of claim 1, wherein the other antiviral agent is a reverse transcriptase inhibitor.

5. The method of claim 4, wherein the reverse transcriptase inhibitor is a nucleoside analog reverse transcriptase inhibitor.

6. The method of claim 5, wherein the nucleoside analog reverse transcriptase inhibitor is abacavir, didanosine, or stavudine.

7. The method of claim 1, wherein the reverse transcriptase inhibitor is a non-nucleoside reverse transcriptase inhibitor.

8. The method of claim 7, wherein the non-nucleoside reverse transcriptase inhibitor is etravirine or rilpivirine.

9. The method of claim 1, wherein the other antiviral agent is a protease inhibitor.

10. The method of claim 9, wherein the protease inhibitor is darunavir.

11. The method of claim 1, wherein the other antiviral agent is an integrase inhibitor.

12. The method of claim 11, wherein the integrase inhibitor is elvitegravir.

13. The method of claim 1, wherein the other antiviral agent is a viral DNA polymerase inhibitor.

14. The method of claim 13, wherein the viral DNA polymerase inhibitor is acyclovir, ganciclovir, or valganciclovir.

15. The method of claim 1, wherein the peramivir, cidofovir, or nevirapine and/or the other antiviral agent is administered orally, intramuscularly, intravenously.

16. The method of claim 1, wherein the individual suffers from a pre-existing health condition correlated with poor prognosis following SARS-CoV-2 disease.

17. The method of claim 16, wherein the pre-existing health condition is selected from the group consisting of cancer, chronic kidney disease, chronic obstructive pulmonary disease, Down Syndrome, heart conditions, heart failure, coronary artery disease, cardiomyopathy, immunocompromised states, obesity, pregnancy, sickle cell disease, smoking, Type T diabetes mellitus, Type 2 diabetes mellitus, asthma, cerebrovascular disease, cystic fibrosis, hypertension, neurologic conditions, liver disease, pulmonary fibrosis, thalassemia, and 65 years or greater of age.

18. The method of claim 1, further comprising administering a second therapy to the individual.

19. The method of claim 18, wherein the second therapy comprises an effective amount of remdesivir, an effective amount of a monoclonal antibody against SARS-CoV-2, mechanical ventilation, or combinations thereof.

20. The method of claim 19, wherein the second therapy comprises administering an effective amount of casirivimab and imdevimab intravenously.
